(19) United States
(12) Patent Application Publication
(10) Pub. No.: US 2005/0095726 A1
(43) Pub. Date: May 5, 2005

(54) Novel high protein tortillas

(76) Inventors: Xiangming Fang, San Diego, CA (US); Lei Huang, San Diego, CA (US); Ping Lin, San Diego, CA (US); Jerald S. Feitelson, San Diego, CA (US); Wei-Wei Zhang, San Diego, CA (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/892,423

(22) Filed: Jul. 14, 2004

Related U.S. Application Data
(60) Provisional application No. 60/487,528, filed on Jul. 14, 2003.

(51) Int. Cl. G01N 33/543

(52) U.S. Cl. 436/518

(57) ABSTRACT

Affinity separation compositions and methods are disclosed for separating targets from complex mixtures. Affinity reagents are bound to a solid support oriented in a manner to facilitate the activity of the affinity reagents which are capable of binding specific targets by affinity recognition. Affinity reagents include IgY antibodies, proteins, peptides, nucleotides and polymers. Targets include proteins, protein-protein complexes, protein-nucleotide complexes, nucleotides, cells and subcellular organelles.

### CROSS-REFERENCE TO A RELATED APPLICATION

This application claims the benefit of U.S. provisional application Ser. No. 60/487,528, filed Jul. 14, 2003.

### FIELD OF THE INVENTION

The present invention relates to affinity separation of biological materials. The invention further relates to compositions of affinity reagents linked to solid supports and the methods that the solid support mediates affinity reagents to separate targets from non-targets in mixtures of biological samples. More specifically, the present invention relates to polyclonal avian IgY antibody compositions and methods of making and using them. The IgY antibodies are covalently bound in an oriented fashion to a solid support via carbohydrates in their Fc region, making the Fab regions of antibodies readily available for reaction with an antigen. The polyclonal IgY antibodies are useful for immunoaffinity capture, separation, purification and detection of a desired protein target in a complex mixture.

### BACKGROUND OF THE INVENTION

The dynamic range of protein concentration spreads from 7 to 8 orders of magnitude in cells and probably up to 12 orders of magnitude in plasma (Corthals, G. L., et al. Electrophoresis 2000, 21, 1104-1115; Anderson, N. L. & Anderson N. G., Mol Cell Proteomics 2002, 1, 845-867). Classical silver-stained two-dimensional electrophoresis gel (2DE) can only display up to four orders of magnitude (Adkins, J. N., et al, Mol Cell Proteomics 2002, 1, 947-955; Gygi, S. P., et al., Proc Nail Acad Sci USA 2000, 15, 9390-9395). This is a significant limitation for the discovery and analysis of important proteins such as regulatory proteins, cytokines, biomarkers, drug targets, etc. Plasma proteome research faces the challenge that the 10-15 most abundant proteins at the mg/ml level are only less than 0.1% of various types of proteins, yet they constitute more than 95% of the mass of total plasma proteins. The important proteins and biomarkers for malignant or non-malignant diseases (e.g. C-reactive protein, osteopotin, prostate-specific antigen, various interleukins and cytokines) are usually at ng/ml to pg/ml levels, making them like needles buried in a huge haystack of abundant proteins (Lopez, M. F., Electrophoresis 2000, 21, 1082-1093; Burtis, C. A. & Ashwood E. R., 2001. Tietz Fundamentals of Clinical Chemistry, 5^{th} Ed., W. B. Saunders Company, Philadelphia). Scientists, especially those who use 2DE and mass spectrometry (MS), require tools or reagents that can specifically separate the abundant proteins and prepare samples for attaining higher sensitivity and better resolution to detect low abundant proteins and thereby “dig deeper into the proteome” (Adkins, J. N., op cit). There is also an unmet need for reference standards for selectively removing high-abundant proteins from plasma to enrich the relatively very low-abundant proteins, thereby increasing their resolution of detection (Zhan, X. & Desiderio D. M., Proteomics 2003, 3, 699-713). Satisfying these demands will make it possible to more precisely measure the low-abundant proteins, particularly in multiplex settings for proteomic profiling (Lee, H., et al., Anal Chem 2002, 74, 4353-4360).

Affinity Separation: Unmet Needs for Proteomic R&D

The Plasma Proteome Project (PPP) of the HUPO (Human Proteome Organization) recognized the challenge of the huge dynamic range of plasma protein concentration in conducting protein discovery research. Therefore, the PPP promoted development of methods and tools for protein fractionation and sample preparation at the inception of the project (HUPO Workshop and Planning Session, Apr. 29, 2002 Bethesda, Md., USA). To best meet the needs of proteomic analysis by 2DE and MS, sample preparation tools for pre-separation or pre-fractionation of proteins should have at least the following features:
1. High specificity and accuracy
2. Low cross-reactivity to other serum proteins
3. Strong avidity (high affinity)
4. High capacity
5. Multiple-species applicability
6. Convenient use
7. High reproducibility
8. Good reusability
9. Minimal disruption to natural condition of sample
10. Reasonable affordability

There are a number of approaches to separate proteins based upon their biochemical and biophysical features such as molecular weight, mass, density, hydrophobicity, surface charge, isoelectric point, tertiary structure, amino acid sequence (epitope), etc. Conventional centrifugation, ultrafiltration, and liquid chromatography, including textile dye ligands (Cibacron Blue), have been previously used to remove albumin and other plasma proteins (Travis, J., et al., Biochem J 1976, 157, 301-306; Rengarajan. K., et al., Biotechniques 1996, 20, 30-32; Butt, A., et al., Proteomics 2001, 1,42-53; Georgiou, H. M., et al., Proteomics 2001, 1, 1503-1506; Tirumalai, R. S., et al., Mol. Cell. Proteomics 2003, 2, 1096-1103; Pieper, R., et al., Proteomics 2003, 3, 1345-1364; Lescuyer, P., et al., Electrophoresis 2004, 25, 1125-1135; Rothemund, D. L., et al., Proteomics 2003, 3, 279-87; Davidsson, P., et al., Rapid Commun Mass Spectrom 2002, 16, 2083-2088). However, these separation processes are not protein-specific and have variable capacity and limited reproducibility.

In comparison, affinity separation is a process specific to selected target proteins. Antibodies, proteins, peptides, nucleotides, etc., are affinity reagents that have been applied for this purpose. Bacterial Protein A and Protein G, which specifically bind to the Fc region of IgG, have been successfully used for specific separation from serum or plasma (Adkins, J. N op cit; Vesterberg, 0. & Anundi H., Appl Theor Electrophor 1991, 2, 159-161). One of the limitations with protein-based affinity reagents, including affinity peptides generated via phage-display, is the limited diversity of products available for various target proteins. Antibody-based separation of proteins, known as immunoaffinity separation, recently became the method of choice due to its specificity and straightforward production. Immunoaffinity separation of proteins using different types of antibodies has generated encouraging data (Burgess-Cassler, A., et al., Clin Chim Acta 1989, 31, 359-365; Kojima, K., J Biochem Biophys Methods 2001, 49, 241-251; Pieper, R., et al., Proteomics 2003, 3, 422-432; Fang, X., et al., in: Reiner, J., et al. (Eds.), Frontiers of Biotechnology and Pharmaceuticals, Vol. 4, Science Press USA, Inc. Monmouth Junction, 2003, pp. 222-245). Commercial kits using either mammalian Immunoglobulin G (IgG) or avian Immunoglobulin Yolk (IgY) have also recently been made available for immunoaffinity depletion of albumin and some other abundant proteins, such as the kits marketed by Agilent, Amersham, Bio-Rad, GenWay Biotech, Pierce, Sigrna-Aldrich, and others.

IgY antibody is immunoglobulin isolated from egg yolks (so called IgY) of the lower vertebrates, such as birds, reptiles, and amphibians (Leslie, G. A. & Clem L. W., J Exp Med 1969, 130, 1337-1352; Hadge, D. & Ambrosius H., Mol Immun 1984, 21, 699-707; Du Pasquier, L., et al., Annu Rev Immunol 1989, 7, 251-275). Avian IgY antibodies have been developed and successfully applied for various types of immunoassays (Larsson, A. & Mellerstedt H., Hybridoma 1992, 11, 33-39; Larsson, A., et al., Poultry Science 1993, 72, 1807-1812; Warr, G. W., et al., Immunol Today 1995, 16, 392-398; Schade, R. & Hlinak A., ALTEX 1996, 13, 5-9; Zhang, W.-W., Drug Discovery Today 2003, 8, 364-371). An outstanding advantage of avian IgY antibodies is that they are secreted by hens into egg yolk, resulting in a high-yielding reservoir of easy-to-access antibodies (Patterson, R., et al., J Immunol 1962, 89, 272-278). Compared to drawing blood, collecting eggs is non-invasive, continuous, convenient, and scalable. One egg yolk contains about 100 mg of total IgY. After a primary injection and three boosts, one hen can produce 40-60 eggs, yielding about 5 grams of antibodies. Distinct from IgG antibodies in molecular structure and biochemical features, IgY antibodies were shown to have several advantages over IgG, particularly for their high avidity and less cross-reactivity to human proteins (Stuart, C. A., et al., Anal Biochem 1988, 173, 142-150; Gassmann, M., et al., FASEB J 1990, 4, 2528-2532; Larsson, A., et al., Clin Chem 1991, 37, 411-414). Unlike IgG, the IgY Fc region does not bind human proteins such as complements, rheumatoid factor, Fc receptor, IgM, etc, significantly increasing IgY's specificity of capture.

Non-IgY Affinity Separation Technologies

For introduction and general comparison, non-IgY affinity separation products for proteomic sample processing are reviewed based upon available published information.

Albumin-Removal Blue-Dye Products

Cibacron® Blue, has been used as a ligand for liquid chromatography and for successfully depleting albumin for nearly thirty years (Travis, J. et al. Biochem J 1976, 157, 301-306). These textile dye materials were further developed into kit products for convenient use. Representative companies that offer this type of product are Millipore (Montage™ Albumin Depletion Kit) and Bio-Rad Laboratories (Aurum). The binding interaction between blue dye and albumin is not based upon specific affinity. While relatively inexpensive, non-specific depletion of other proteins is the major weakness of this technology. Sigma-Aldrich, using a type of Proprietary Blue Matrix (ProteoPrep™ Blue Albumin Depletion Kit), claims low non-specific binding because it does not contain Cibacron® Blue.

IgG-Removal Protein-Sorbent Products

After albumin, the second most abundant protein in serum or plasma is IgG. Many vendors supply Protein A or Protein G sorbents, bacterial proteins that specifically bind the Fc region of IgG (Kronvall G. et al. J Immunol. 1970, 104, 140-147). This class of products is distributed by Sigma, Bio-Rad, Agilent, Applied Biosystems and Amersham (see below).

Though not yet extensively used in proteomic studies, recombinant Protein L was cloned from Peptostreptococcus magnus and distributed by Affitech AS (Oslo, Norway). This protein binds the kappa light chain of antibodies from many species without interfering with their antigen binding sites.

BioSepra™, the Process Division of Ciphergen Biosystems, has developed MEP HYPERCEL, an alternative to Protein A or Protein G for process-scale purification of recombinant antibodies and antibody fragments from many species. These sorbents may also be used in applications for the capture and separation of IgG antibodies in plasma or serum.

Polyclonal Goat IgG Antibody Products

This group of products applies polyclonal Goat IgG antibodies against different target proteins for immunoaffinity binding and separation. Representatives include Amersham Biosciences (Ettan™ Albumin and IgG Removal Kit), Bio-Rad (Aurum Serum Protein Mini Kit), Applied Biosystems (POROS® Affinity Depletion Products) and Agilent Technologies (Multiple Affinity Removal Systems, MARS). These products contain Protein A or G either as a sorbent for direct binding IgG in plasma or serum, or as a linker for conjugating the polyclonal goat capture antibodies to the microbead surface.

These polyclonal goat IgG capture antibodies are affinity-purified and coupled through Protein A conjugated to the solid phase matrix. Their capacity (˜2 mg HSA per ml packed bed volume) is typically lower than the blue dye-based products, but they have much greater specificity for human serum albumin. In addition, depending on the plasma/serum loading, percentage albumin removal is also higher than with dye-based products, yielding a substantially superior product. For many sensitive proteomics applications, such as Multidimensional Protein Identification Technology (MudPIT) (Washburn M. P. et al. Nat Biotechnol. 2001, 19, 242-247), reducing HSA levels from the most abundant protein before depletion (˜60%) to the second or third most abundant protein after depletion (˜6%) is insufficient for direct analyses because of the very high backgrounds still evident.

The same point applies for IgG removal reagents, where >99% removal is ideal because of the immunoglobulins' very high initial abundance and their great molecular heterogeneity. Agilent's MARS use Protein A or Protein G for this purpose. Amersham and Bio-Rad offer prepacked spin columns that are convenient to use and only require a centrifuge and standard reagents and collection tubes. Agilent offers the most advanced application of IgG-based solutions by packing an HPLC column with POROS® 20 beads coupled to goat IgGs specific to HSA, IgG, Transferrin, α1-Antitrypsin, Haptoglobin and IgA. Standard LC fittings are provided. The user must have an LC system available to use Agilent's MARS columns. Complete kits are available, including two proprietary buffers, and filtration and concentration spin columns. About 85% of human serum proteins are removed, with very high-level capture of the target proteins. The columns are extensively reusable if handled properly. One drawback to this system is that urea is added to the extraction buffer which precipitates at low temperatures, requiring room temperature protein concentration for analyzing bound material.

Divide and conquer is a strategy that has been articulated to cope with the overwhelming dynamic range of protein concentrations present in proteomes, which is usually several orders of magnitude beyond the detection range of the currently most useful protein separation and identification methods, such as 2DE, LC and MS. For analytical management, the human proteome must be divided into sub-proteomes, and a complex protein mixture must be fractionated, in order to accurately separate and measure target proteins. Affinity separation is one of the most specific and effective approaches for fractionation of protein mixture or complex biological solution. Table 1 provides an overview, summarizing representative technologies and products.

TABLE 1
Technology and Product Comparison Summary

| Comparison | Technology 1 | Technology 2 | Technology 3 | Technology 4 | Technology 5 |
|---|---|---|---|---|---|
| Name and | Aurum ™ Serum | ProteoPrep ™ | Albumin and | Multiple Affinity | IgY Microbeads of the |
| Features of | Protein Mini Kit | Blue Albumin | IgG Removal | Removal System | Present Invention |
| Technologies | | Depletion Kit | Kit | | |
| Antibodies | none | none | Goat IgG | Goat IgG against | Avian IgY against HSA, |
| | | | against HSA | HSA, IgG, | IgG, IgA, IgM, Transferrin, |
| | | | | Transferrin, α1- | Fibrinogen, Haptoglobin, |
| | | | | Antitrypsin, IgA, | ApoA-I, ApoA-II, α1- |
| | | | | Haptoglobin | Antitrypsin, α1-Acid |
| | | | | | Glycoprotein, α2- |
| | | | | | Macroglobulin |
| Ligands | Cibacron Blue | Blue Dye and | Protein A or G | Protein A | No Protein A or G |
| | and Protein A | Protein G | | | |
| Microbeads | Resin | Agarose | Resin | POROS ® 20 | UltraLink Hydrazide Gel |
| Product Form | Spin Column kit | Spin Column | Spin Column kit | Pre-Packed | Microbead Slurry; Spin |
| | | kit | | HPLC Column | Column kits, and pre-packed |
| | | | | kit | FPLC columns |
| Capacity | 50 μl | 75 μl | 10-15 μl | 20-100 μl, | 10-175 μl, depending on |
| (Serum or | | | | depending on | target(s) |
| Plasma per ml | | | | column types and | |
| of product) | | | | sizes | |
| Target Protein | <90% | 80-95% | >95% | 98-99% | 95-99.5% |
| Removal | | | | | |
| Unique | No antibodies | Proprietary | Good albumin | Multiple reusable | Diversified product types, |
| Features | | Blue Matrix | specificity | | directly applicable to |
| | | | | | animals, mouse, rat, dog, etc. |
| Pros | Inexpensive, | Inexpensive, | Inexpensive, | Well-developed | Convenient and specific |
| | convenient to | better | convenient to | and having | removal of 12 abundant |
| | use | specificity than | use | multiple target | serum proteins in one step. |
| | | Cibacron Blue, | | capacity. New | >20-fold enhancement of |
| | | low sample | | column available | low abundant proteins. All |
| | | dilution | | against mouse | antibody microbead products |
| | | | | Albumin, IgG & | available separately. |
| | | | | Transferrin. | |
| Cons | Incomplete | Incomplete | Low capacity | Proprietary | Relatively lower capacity as |
| | capture at | capture at | | buffers. Some | MIXED12 in spin column |
| | specified | specified | | IgGs unsuitable | format |
| | capacity. High | capacity | | for column | |
| | non-specific | | | recycling, e.g. | |
| | protein capture | | | against ApoA-I, | |
| | | | | IgM, and α2- | |
| | | | | Macroglobulin | |

The present invention has a great potential for use on other body fluids, subcellular fractions, tissue and cell culture extracts, and other sub-proteomes. The technology is readily adaptable to different formats and scales of protein separation by using suitable devices or carriers. The unique biochemical and immunological features of this type of material enable its further development. The present invention can also be combined with other protein fractionation products to better meet the needs of scientists and provide solutions to facilitate protein target discovery and validation.

### BRIEF SUMMARY OF THE INVENTION

Briefly, in a specific embodiment of the present invention, an affinity separation composition is provided which comprises affinity reagents linked to a solid support and the methods that the solid support mediates affinity reagents to separate targets from non-targets in mixtures of biological samples. A preferred embodiment of the affinity reagents employed in the present invention is a polyclonal antibody composition of Immunoglobulin Yolk (IgY antibody) having an Fc region and an Fab antigen binding regions. The IgY antibody composition comprises a solid support covalently linked to oxidized glycosylation moieties in the Fc region of the polyclonal IgY antibodies wherein the Fab regions of the IgY polyclonal antibodies are capable of reacting with an antigen. The present invention also includes the above described polyclonal IgY antibody composition that additionally contains an antigen bound or hybridized to the Fab antigen binding regions of the antibody.

The present invention additionally includes a method of preparing the polyclonal IgY antibody compositions which comprises contacting reactive polyclonal IgY antibodies, wherein the glycosylation moieties in the Fc region have been oxidized, with a solid support material containing reactive moieties wherein the oxidized glycosylation moieties of the polyclonal IgY antibodies covalently bond with chemically reactive moieties of the solid support material by forming covalent bonds whereby the IgY polyclonal antibodies are oriented to allow the Fab regions to react with an antigen.

The present affinity separation compositions are used as affinity binding reagents to capture separate, and detect one or more targets (proteins, antigens, or other biological materials) from a complex mixture. Using IgY antibody composition as an example, this affinity separation process can be generally accomplished by:
a. providing a complex target (protein or antigen) mixture;
b. contacting the complex target (protein or antigen) mixture with the present IgY polyclonal composition of the present invention whereby a desired target in the complex mixture binds with the IgY polyclonal antibodies in the Fab regions; and
c. recovering the treated complex target (protein or antigen) mixture wherein the concentration of the desired target (protein or antigen) has been substantially reduced for depletion or substantially enriched for affinity separation.

The complex target (protein or antigen) mixture can be plasma, serum, cerebrospinal fluid, urine, pulmonary alveolar lavage, vitreous humor, nipple aspirates, tissue samples, cell extracts or industrial streams from cell cultures. Additionally, the desired target (protein or antigen) that has specifically bound to the affinity reagents can be recovered and studied or analyzed to determine if other targets (protein or antigen) or compounds (e.g., lipids, hormones, etc.) in the complex are associated with the desired target.

Of particular interest in practicing the present invention, the major proteins present in serum are immunodepleted by contacting the serum with the present polyclonal IgY composition wherein the polyclonal IgY antibody is reactive with a major protein present in the serum. For example, human serum albumin (HSA) and IgG constitute approximately 75% of all proteins present in human serum. To eliminate HSA from serum, the serum would be contacted with the present polyclonal IgY antibody composition that contains anti-HSA IgY antibodies covalently conjugated to a solid surface, such as microbead carriers. Similarly, to eliminate IgG from the serum, the serum would be contacted with the present polyclonal IgY antibody composition that contains anti-IgG antibodies. Elimination of the predominant proteins is desirable because it makes detection and analysis of function of other proteins present in minor amounts easier in the depleted serum.

The polyclonal IgY compositions of the present invention, directed against Albumin, IgG, Transferrin, α1-Antitrypsin, IgA, IgM, α2-Macroglobulin, Haptoglobin, Apolipoproteins A-I and A-II, Orosomucoid (α1 Acid Glycoprotein) or Fibrinogen, have all of the following advantages cited earlier:
High specificity for their targets;
High antigen-binding capacity (avidity) compared to other antibody-based products;
The same reagents are often applicable to multiple-species. Anti-human protein IgY antibodies often have a broad host range, with excellent binding of orthologous proteins from other mammalian species compared to IgG antibodies raised in rabbits, mice or goats, due to the great evolutionary distance between chickens and mammals;
Results are highly reproducible;
The compositions have good reusability; they can be recycled with little or no loss of antigen-binding specificity or capacity even after more than 20 uses;
There is minimal disruption to the natural condition of biological samples;
The compositions are convenient to use in a variety of formats, including preparative-scale Liquid Chromatography (LC) columns, spin columns, packed plugs in small tips, magnetic or paramagnetic micro- or nano-particles, or microfluidics devices;
The costs are reasonable, with the products generally affordable;
The materials can be made in large quantities due to efficiencies of production.

In another aspect of the present invention, the present polyclonal IgY antibody composition is made with anti-Fibrinogen IgY antibodies and this composition is used to deplete Fibrinogen (Coagulation Factor 1) from plasma. This will allow for proteomic analysis of the plasma proteins without the extensive proteolysis induced by standard methods of clotting. Thus, plasma proteomics analyses can be carried out with much greater precision than previously possible. The present polyclonal IgY antibody compositions can be employed to affinity-deplete high-abundant plasma and serum proteins that are present at levels above 1.0 mg/ml. Removing high-abundant proteins will enable researchers to effectively analyze low-abundance plasma proteins. This is particularly significant for detecting extremely low-level proteins at early disease stage, those induced by various drug treatments, toxicity detection, and for conducting multiplex protein profiling.

The polyclonal IgY compositions of the present invention can be used in high-throughput sample processing equipment, such as Applied Biosystem's BioCad Vision system. For example, see “Novel Plasma Protein Separation Strategy Using Multiple Avian IgY Antibodies For Proteomic Analysis”, in Methods in Proteomics (Smejkal G. ed. 1994), which is incorporated herein by reference. This format is widely used by industrial-scale proteomics companies and has sophisticated, computer controlled sample handling capabilities with adjustable flow rates, various sized cartridge volumes, in-line pH monitoring and elution profiles.

### BRIEF DESCRIPTION OF THE FIGURES

FIG. 1—Basic composition and process of affinity separation. Listed are the various elements, components and materials that can be used to enable the composition and process of affinity separation of specific targets from mixture containing non-specific targets.

FIG. 2—Variations of basic compositions of affinity separation. Diagrams depict two examples of variations of the basic composition and process shown in FIG. 1. A, shown is to use the molecular affinity bridge, e.g. biotin and avidin or streptavidin, to link affinity reagent to solid support. B, multiple affinity reagents (e.g. IgY antibodies) mixed in certain ratio first, then linked to solid support, different from that in FIG. 1, where one affinity reagent linked to solid support.

FIG. 3—Comparison of one-round versus two-round depletion of HSA (FIG. 3 illustrates the depletion efficiencies using two sequential columns.)

FIG. 4—Initial capacity measurement of anti-HSA Microbeads. SDS PAGE analysis of HSA depletion in human serum samples. A: 4 μl serum; B: 10 μl serum.

FIG. 5—Repeated capacity measurement of anti-HSA Microbeads. 25 μl of diluted human serum (1:10 dilution in TBS to obtain concentration of 8 mg proteins/ml or 1:5 dilution in TBS to obtain concentration of 16 mg proteins/ml) were subjected to 2 rounds of HSA depletion by means of 25 μl IgY microbeads (conjugation ratio: 5 mg IgY/ml microbeads). A. HSA was completely removed from serum. The bound proteins are mainly albumin. Results shown are 3 μl/lane of pooled materials from 4 experiments. B. HSA was completely removed in 1:10 diluted serum, and 90% depletion was observed when serum was 1:5 diluted.

FIG. 6—Depletion of IgG by IgY Microbeads. Shows results wherein 50% of IgG-Fc was depleted for the samples at protein concentrations of 5 mg/ml. 80% depletion was observed for the samples at 2.5 mg/ml and 1.25 mg/ml. The negative control unconjugated microbeads failed to bind to IgG-Fc.

FIG. 7—Depletion of Apolipoprotein A-I by IgY Microbeads. Shows results wherein about 95% of Apolipoprotein A-I was depleted after 4 rounds of depletion.

FIG. 8—Protein Separation Capacity of IgY Microbeads for HSA. Shows the capacity of anti-HSA microbeads to be approximately 2.4 mg of HSA bound per ml of packed bed volume.

FIG. 9—Separation of Individual Target Proteins by IgY Microbeads. Shows the sequential depletion of four human proteins using individual IgY microbead gels, with virtually no cross-reactivity between non-targeted abundant proteins.

FIG. 10—Test of Separation Efficiency of MIXED6. The two-spin column system effectively removes all 6 target proteins (HSA, IgG, Fibrinogen, Transferrin, IgA and IgM) from human plasma.

FIG. 11A—Depletion of Human Plasma Using MIXED12. The one-spin column system effectively removes all 12 target proteins (HSA, IgG, Fibrinogen, Transferrin, IgA, IgM, Apolipoprotein A-I, Apolipoprotein A-II, Haptoglobin, α1-antitrypsin, α1-Acid Glycoprotein and α2-Macroglobulin) from two different pooled human plasma samples.

FIG. 11B—Depletion of Human Serum Samples Using MIXED12. The one-spin column system effectively removes all 12 target proteins (HSA, IgG, Fibrinogen, Transferrin, IgA, IgM, Apolipoprotein A-I, Apolipoprotein A-II, Haptoglobin, α1-antitrypsin, α1-Acid Glycoprotein and α2-Macroglobulin) from three different human clinical serum samples.

FIG. 12—2-Dimensional Electrophoresis of Human Serum Sample Treated by MIXED12. Direct evidence is provided for effective removal of the targeted abundant proteins in human serum.

FIG. 13—2-Dimensional Electrophoresis of Human Plasma Sample Treated by MIXED12. Direct evidence is provided for effective removal of the targeted abundant proteins in human plasma.

FIG. 14—Analysis of Recyclability of IgY Microbeads. Shows recycling of anti-HSA twenty times with no loss of capacity or specificity

FIG. 15—Analysis of Recyclability of MIXED12 Spin Column. Shows recycling of MIXED12 twenty times with no loss of capacity or specificity

FIG. 16—Serial Depletion of 8 Mouse Plasma Proteins by IgY Microbeads. Shows effective sequential depletion of at least 7 of the 8 orthologous mouse proteins using anti-human protein IgY microbeads.

FIG. 17—Comparison of anti-HSA and Anti-BSA IgY Microbeads. Panel A: anti-HSA IgY microbeads; Panel B: anti-BSA IgY microbeads. Consistent with Table 5, significant differences are shown between the cross-species albumin binding capacities (human, bovine, mouse and rat) of anti-HSA IgY microbeads and anti-BSA IgY microbeads.

### Brief Description of the Tables

Table 1—Technology and Product Comparison Summary

Table 2—Efficiency of anti-HSA IgY microbeads for “spiked” samples in PBS using different antibody loading densities on microbeads

Table 3—MIXED IgY Microbead Products

Table 4—Depletion Efficiency of MIXED12 after Multiple Cycles

Table 5—Depletion Efficiency of Anti-HSA and BSA IgY Microbeads

### DETAILED DESCRIPTION OF THE INVENTION

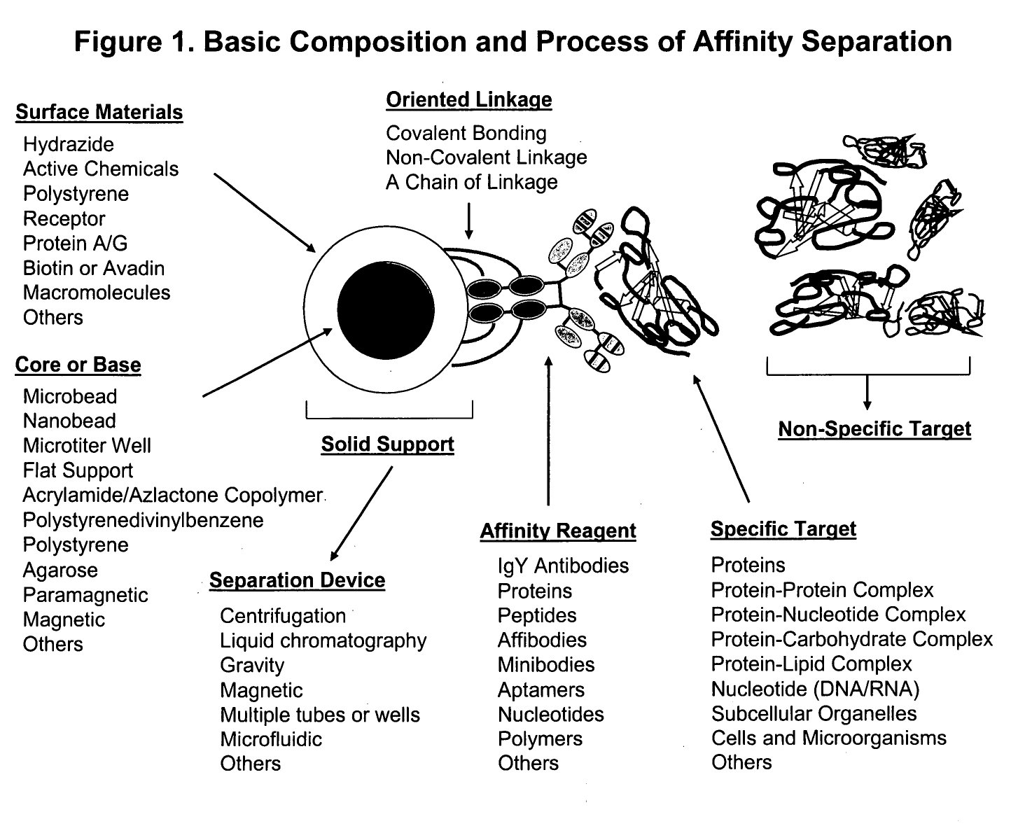

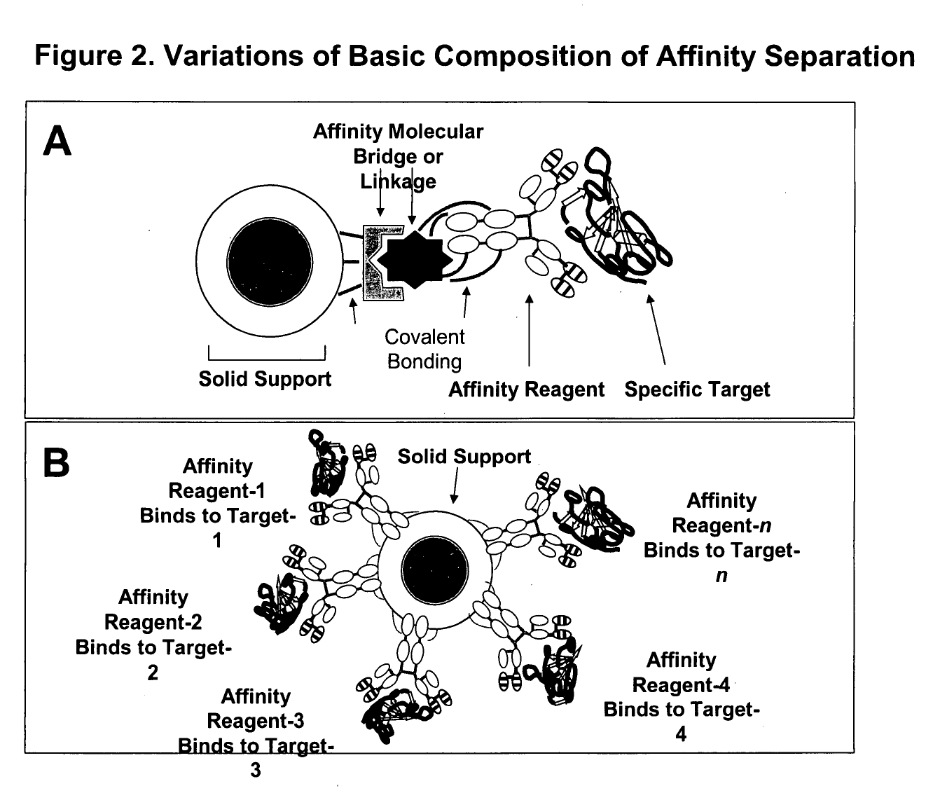

In practicing the present invention, the following basic components, processes, and variations (FIGS. 1 and 2) are employed to conduct an affinity separation process:

Affinity Reagents —These are biological substances or macromolecules that can specifically bind to targets through affinity recognition and attractive forces between reagents and targets. Affinity recognition, resembling the relationship between lock and key, is highly specific for the target and usually has a dissociation constant below 10⁻⁸ M (Winzor D. J., J Chromatogr. 2004 1037(1-2): 351-67; Chaiken I. M., J Chromatogr. 1986, 376: 11-32). The affinity reagents can include IgY antibodies, proteins, peptides, affibodies, minibodies, aptamers, nucleotides, polymers and others.

Specific Targets —These are also biological materials, macromolecules, molecules, or complexes. The specific targets are usually antigens that can induce antibodies in animals. The specific targets can also be other materials such as proteins, protein-protein complexes, protein-nucleotide complexes, protein-carbohydrate complexes, protein-lipid complexes, nucleotide (DNA/RNA), subcellular organelles, cells and microorganisms and others. The specific targets are usually mixed or complexed with other non-specific targets. The specific targets that bind specifically to affinity reagents can be separated from those non-specific targets in a given mixture of specific targets and non-specific targets.

Oriented Linkage —These are the chemical or biological materials that can link affinity reagents to the surface of solid supports. Linkages can be covalently bonding between the affinity reagents and the surface of the solid support. Linkages also can be indirect, through a chain of covalent bonding and non-covalent affinity binding, as shown in FIG. 2.

Solid Support —These are the materials that are attached to the affinity reagents through oriented linkage and can mediate the affinity reagents to separate bound targets from those non-specific targets. The solid support generally comprises surface materials and a core or base. The surface materials are the active chemical or biological materials that can link the solid support to the affinity reagents. These materials comprise hydrazide, active chemicals, polystyrene, receptor, protein A/G, biotin, avidin, strepavidin, macromolecules and others. The core or base is coated with the surface materials and linked to affinity reagents via surface materials. The core or base can be the materials that help or mediate the separation of that affinity reagent-target complex. Examples of the core or base include microbeads, nanobeads, microtiter wells, flat supports, acrylamide/azlactone copolymer, polystyrenedivinylbenzene, polystyrene, agarose, paramagnetic, magnetic and others.

Separation Devices —These are the forces, attractions, apparatus, or processes that mediate the separation of the affinity reagent-target bound solid support from mixture of targets or biological materials. Examples of the separation devices include gravity, centrifugation, liquid chromatography, magnetic force, multiple tubes or wells, microfluidic and others.

The basic composition and process of affinity separation specified in the present invention are depicted in FIG. 1 with some examples of related materials. The composition and process can be engineered into different variations. FIG. 2 depicts two classes of variations:

Variation 1−Shown in FIG. 2A, the linkage of affinity reagents to solid support is indirect, which is designed to have a molecular bridge, a pair of affinity reagents such as biotin and avidin. Each end of the molecular bridge is fixed to the affinity reagent or solid support through covalently bonding. This is a type of chain linkage, where the linkers can be combinations of covalent or non-covalent associations.

Variation 2−The solid support can be attached to affinity reagents in a different way. Shown in FIG. 2B, the solid support is bound to a group of affinity reagents mixed at a given ratio before the linkage process takes place. The ratio of mixing of affinity reagents is based upon the optimized binding of the affinity reagents to targets and effectiveness of affinity separation.

In one embodiment of the present invention, an affinity separation composition for separating one or more target compounds present in a complex mixture is made by linking an affinity reagent to a solid support oriented in a manner to facilitate the activity of the affinity reagents or its ability to further react with a target. Once prepared the affinity separation composition is contacted with a complex mixture to remove the target from the mixture by affinity recognition of the target by the affinity reagent. The solid support component of the affinity separation composition mediates the separation of the affinity reagent-target complex from the complex mixture. The resulting complex mixture has a reduced level of the target and preferably no detectable levels of the target. The affinity reagent-target complex can then be processed to strip the target so that the affinity separation composition can be re-used. Additionally, the target can be recovered and/or analyzed to determine if there is an association between other materials and the target.

More specifically, in practicing the present invention, polyclonal IgY antibodies can be covalently conjugated to a solid support material by oxidizing the glycosylation moieties in the Fc region of the polyclonal IgY antibodies and then reacting oxidized antibodies with a solid support material that has reactive moieties that will form a covalent bond (conjugation) with the oxidized glycosylation moieties. This reaction forms an antibody composition that orients the antigen binding region away from the support material and allows the antibody to react with an antigen.

While the exact ratio of IgY antibody to solid support material is not critical, typically 5, 10, 15, or 20 mg of IgY antibody are reacted with 1 ml of solid support material. The solid support material can be of any desired shape, size or physical configuration such as microbeads, membranes, chip surfaces and the like. Any shape having a large surface area is preferred. The chemistries involving the oxidation and conjugation reactions are well known to one of ordinary skill in the art.

The solid support may contain a spacer arm that reduces steric hindrance and allows the orientation of the antibody so that the Fc region is positioned toward the support and the Fab regions are positioned away from the support where it can more readily react/bind with an antigen. A support material and spacer arm with minimal nonspecific binding characteristics is preferred. The specific length of the spacer arm is not critical and spacer arms can be up to 23 atoms or longer if desired.

The solid support can be in any physical configuration such as for example beads or membranes. However, any configuration that increases the surface area of the solid support is preferred because an increased surface area will allow for more attachment sites of the IgY antibody in a given volume. For this reason beads, including nanobeads, are a preferred solid support configuration. Beads can be in a pre-packed or batch mixture format. Beads can also be used in a continuous process format. Magnetic and paramagnetic beads can be also be employed as the solid support to aid in the separation of the polyclonal IgY beads after being contacted with the complex protein mixture being immunodepleted.

If a solid support material is used that will react specifically with the Fab regions of the IgY antibody then the support material can be coated to render the material non-reactive to the Fab regions and facilitate a reaction with the Fc region of the IgY antibody. For example, polystyrene beads, including styrene nanobeads, can be coated with avidin or streptavidin to prevent reactions between the polystyrene and the Fab regions on the antibody. The avidin coated polystyrene beads are then reacted with biotin that has been modified to contain hydrazide groups that can then react with the Fc region of the IgY antibody. This allows for the proper orientation of the IgY antibody for maximum efficiency in hybridizing with the desired protein (antigen) in the complex protein mixture. In another example, periodate-oxidized IgY is reacted with a bifunctional linker molecule containing a hydrazide at one end and a ligand at the other end. The resulting IgY-ligand molecule then binds tightly and specifically with a ligand receptor bound to a solid surface, such as a microbead. The linker molecule is bifunctional and comprises an hydrazide moiety at one end to bind to the Fc region of the IgY and biotin at the other end which serves as the ligand to bind to the solid support. Coupling of the biotinylated IgY to a solid surface is mediated through avidin or streptavidin which coats the underlying solid surface.

Once the polyclonal IgY antibody composition (collectively referred to hereinafter as “present IgY composition”) is prepared, it can be used to immunoprecipitate a desired protein from a complex protein mixture. This is done by contacting or incubating a sample of the complex protein mixture with the present IgY composition. The depleted sample can be recovered and contacted with a fresh or recycled batch of the present IgY composition one or more additional times depending on the binding capacity and protein concentration of the sample. The sample is then analyzed to determine if all of the desired protein has been removed from the sample. Additionally, after the depletion is complete, the present IgY composition used in the depletion reaction can be treated to strip the desired protein from the antibodies, which can then be analyzed to determine if other proteins or materials are associated with the desired protein.

The exact amount of polyclonal IgY antibody composition used in practicing the present invention (immunodepletion process) is not critical as any available IgY antibody will react with the target protein. Excess amounts of IgY antibody are employed if all of the target protein is to be removed from the complex protein mixture. If less than all of the target protein is to be removed from the complex protein mixture then the amount of IgY antibody is adjusted accordingly. Routine titration experiments can be conducted to determine the optimum amount of antibody needed per weight of target protein.

In human serum depletion with the present polyclonal IgY composition it is desirable to remove at least about 95% by weight and preferably at least about 98% of the high abundant proteins. For HSA removal at least about 99% and preferably at least about 99.9% or more is removed from the complex protein mixture.

When mixtures of different IgY antibodies are used to deplete multiple target proteins from a complex mixtures the ratio of the different IgY antibodies should preferably approximate the ratio of the target proteins present in the complex protein mixture. As mentioned above, routine analytical procedures (ELISA, Western blot, etc.) are employed to determine the ratio of target proteins present in the complex protein mixture and then the corresponding IgY antibody ratios are calculated and mixed accordingly. For example, if HSA and IgG are the target proteins in a serum sample and upon analysis of the serum sample are present in a weight ratio of 4:1 (HSA/IgG) then it would be preferred to employ an IgY antibody composition that contain about 80% anti-HSA IgY antibodies and 20% anti-IgG IgY antibodies (4:1 ratio) in amounts effective to react with substantially all of the target proteins present in the complex mixture. If other target proteins are to be removed from the serum then the ratios of all of the target proteins are calculated and the specific IgY antibodies are prepared in accordance to the calculated protein ratios.

The following terms are defined for use herein:

“IgY polyclonal antibody” means gamma globulins derived from the egg yolk of an avian species.

“Avian species” refers to any bird, preferably chickens (Gallus gallus).

“Covalently linked” when referring to IgY antibodies means oriented conjugation of the IgY antibodies with the antigen binding fragment available for antigen binding. This occurs by oxidizing the IgY-Fc glycosylation moieties, converting hydroxyl groups to reactive aldehyde groups, which then react with chemical groups on the solid support forming stable covalent bonds.

“Antigen” means any compound that is recognized and specifically bound by the polyclonal antibody preparation. Typically, this same antigen is used to immunize the bird for producing polyclonal antibodies in the yolk. The immunization is typically done by injecting a bird with a purified antigen. In the case of protein antigens, a bird can be injected with polynucleotides that can express the protein antigen or immunogenic portions thereof thereby making the antigen in situ in the bird.

The present invention is particularly useful in depleting abundant proteins present in plasma, serum and other body fluids and tissue samples to allow for a more accurate quantitation of less abundant proteins present in those materials. Abundant proteins present in serum include, but are not necessarily limited to, human serum albumin (HSA), IgG, Transferrin, IgA, α2-Macroglobulin, IgM, α1-Antitrypsin, Complement C3, Haptoglobin, Apolipoprotein A-I, Apolipoprotein A-II, Apolipoprotein B, and α1-acid glycoprotein (Orosomucoid). In addition to these highly abundant proteins, plasma also contains Fibrinogen and other clotting factors.

To deplete serum or plasma of any one or more of these abundant proteins, polyclonal IgY compositions of the present invention are prepared using an antibody that will hybridize to the desired protein to be depleted. The serum or plasma sample is contacted with that specific IgY composition to remove the desired protein. Preferably, all proteins present in plasma and serum in an amount of 1.0 mg/ml or greater are immunodepleted according to the present invention. The process can be repeated to remove additional proteins. Alternatively, two or more antigen specific polyclonal IgY compositions can be combined and then several proteins can be depleted in a one step process.

Other applications of the present IgY polyclonal antibody compositions include their use in IgY antibody arrays, IgY antibody microbeads that will hybridize with any desired protein whether it is an abundant protein or not, IgY antibody columns and IgY antibody diagnostic applications.

IgY antibodies are made in birds and preferably chickens. The birds are injected with the purified protein (desired protein to be removed from the complex protein mixture) that acts as an antigen in the bird resulting in the production of IgY antibodies that will bind with the protein. This produces high affinity antibodies with high avidity. Gene-specific IgY antibodies can also be made by injecting gene expression vectors where the antigenic protein is made in situ. IgY is then collected from the yolks of bird eggs employing standard separation techniques. See Drug Discovery Today, Vol 8, No 8, 2003, 364-371, which is incorporated herein by reference.

The IgY antibodies specific for the desired protein are separated from the other IgYs by antigen affinity purification employing similar procedures to the antigen affinity purification of IgG. See The Journal of Cell Biology, Volume 141, Number 7, Jun. 29, 1998 pp. 1515-1527, which is incorporated herein by reference. For affinity purification of anti-HSA IgY, purified HSA is coupled to cyanogen bromide-activated Sepharose 4B (Pharmacia Biotechnology, Inc.). The Total IgY preparation is then contacted with the HSA-bound Sepharose 4B, wherein the anti-HSA antibodies specifically bind to the antigen on the Sepharose beads. After washing to remove the non-specific IgY antibodies, the anti-HSA antibodies are then eluted sequentially with 0.1 M glycine-HCl (pH 2.5) and the column neutralized with 0.1 M triethylamine (pH 11.5) before reequilibration. The affinity purified IgY antibodies are then used in a reaction with the reactive solid support material to make the present polyclonal IgY compositions.

In one embodiment, the purified IgY antibodies are oxidized in the Fc glycosylation region with sodium metaperiodate, followed by dialysis to remove the oxidizer. The oxidized IgY antibodies are then reacted with azlactone-acrylamide copolymer microbeads (Pierce UltraLink® Hydrazide Gel), which is an affinity support for immobilizing glycoproteins through oxidized sugar groups. A preferred bead diameter is in the range of from about 50 to about 80 μm with an average diameter of about 60 μm. It is ideal for immobilizing IgY polyclonal antibodies since they contain abundant carbohydrates located on the Fc portion of the antibody molecule. Because such antibodies are coupled to the UltraLink® Hydrazide Gel through the Fc portion only, they are properly oriented with their antigen-binding sites unobstructed, offering greater antigen binding capacity. In order to optimize orientation of the antigen binding region for optimal antigen binding capacity the following spacer arm is employed with the azlactone-acrylamide copolymer microbeads:

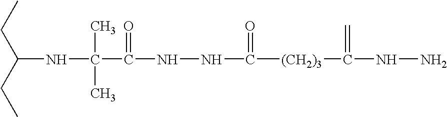

The immobilization chemistry uses sodium periodate to oxidize glycoproteins, converting vicinal hydroxyl groups in sugars to reactive aldehyde groups. The aldehydes then react with hydrazide groups on the UltraLink® Hydrazide Gel to form stable hydrazone bonds. The coupling conditions are flexible with regard to time and temperature. A long (23-atom) spacer arm that reduces steric hindrance and a support with minimal nonspecific binding characteristics makes this a favorable gel for affinity chromatography. The protein-coupled columns may be regenerated and reused at least 20 times under the proper stripping and regeneration conditions.

UltraLink® Biosupport Medium is hydrophilic, charge-free, high-capacity, highly cross-linked, rigid, copolymeric and porous. This means that the support has minimal nonspecific interactions with the sample. The porosity, rigidity and durability of this support are important considerations when working with large volumes of samples requiring fast-flow techniques and large-scale applications. Agarose supports are extremely useful for gravity flow procedures; however; a more rigid support is required if pressures are greater than 25 psi. UltraLink® Biosupport Medium is useful for medium-pressure techniques. When packed into a 3 mm inside diameter×14 cm height column, UltraLink® Supports have been run to approximately 400 psi (system pressure) with no visual compression of the gel or adverse effects on chromatography. Typically these columns can be used with linear flow rates of 85-3,000 cm/hour with excellent separation characteristics. See, for example, Brown, M. A., et al. (2000). Identification and purification of vitamin K-dependent proteins and peptides with monoclonal antibodies specific for gamma-carboxyglutamyl (Gla) residues. J. Biol. Chem. 275(26), 19795-19802; Coleman, P. L., et al. (1988). Affinity chromatography on a novel support: azlactone-acrylamide copolymer beads. FASEB J 2: A1770 (#8563); Coleman, P. L., et al. (1990). Azlactone copolymer beads: applications in bioseparations. J. Cell. Biochem. 44, 19 (S14D); Milbrath, D. S., et al. (1990). Azlactone-functional supports useful in affinity chromatography and other bioseparations. AICHE Extended Abstracts #104E; Milbrath, D. S., et al. (1989). Azlactone polymer supports for bioseparations. ACS Abstracts; Rasmussen, J. K., et al. (1991/1992). Crosslinked, hydrophilic, azlactone-functional polymeric beads: a two-step approach. React. Polym. 16, 199-212; Rasmussen, J. K., et al. (1992). Mechanistic studies in reverse-phase suspension copolymerization of vinyldimethylazlactone methylenebis (acrylamide). Makromol. Chem., Macromol. Symp. 54/55, 535-550; Rasmussen, J. K., et al. (1990). Hydrophilic, crosslinked, azlactone-functional beads-a new reactive support. Polymer Reprints 31(2), 442-443; U.S. Pat. No. 4,871,824 (Heilmann, et al.); and European Patent Publication 0 392,735 A2 all of which are incorporated herein by reference.

Serum protein depletion can be achieved by loading 50 μl of anti-HSA IgY azlactone-acrylamide copolymer microbead slurry (25 μl beads) onto a Handee Mini-spin Column (Pierce, Prod # 69705) and inserting the column in an Eppendorf tube, which is centrifuged for 8 seconds at full speed to remove the solution. Then 25 μl of serum (recommended 6- to 10-fold dilution of serum) is added to the dried microbeads and incubated at room temperature for 30 min. The microbeads should be resuspended once every 5 minutes with gentle stirring using a Pipetman tip. After incubation, the column is inserted into a clean Eppendorf tube and centrifuged for 8 seconds at full speed. The collected sample is subjected to another round of depletion as described above. The obtained sample is ready for further study and/or further depletion of another protein, such as IgG, employing a specific anti-IgY covalently conjugated to azlactone-acrylamide copolymer microbeads. Likewise other proteins can be depleted if desired.

In another embodiment of the present invention, the protein that is immunoprecipitated onto the polyclonal IgY compositions of the present invention can be analyzed to determine if there is an association between the immunopreciptated protein and any other proteins or other compounds, such as lipids, carbohydrates, hormones and the like, present in the serum. To analyze protein bound to IgY microbeads, the microbeads are washed 2× with 0.5 ml TBS and then eluted with 25 μl of 100 mM glycine-HCl pH 2.5. The collected sample is then neutralized with 2.5 μl of 1M Tris-base pH 8 and is then ready for analysis.

Following are examples that illustrate procedures for practicing the invention. These examples should not be construed as limiting. All percentages are by weight and all solvent mixture proportions are by volume unless otherwise noted.

### EXAMPLE 1

### Direct Covalent Conjugation of Individual IgY Antibodies to Solid Support

IgY microbeads were initially developed by optimizing conditions for covalently conjugating affinity-purified anti human serum albumin (HSA) IgY to UltraLink® Hydrazide Microbeads (Pierce Biotechnology, Rockford, Ill., USA) at different antibody-microbead conjugation ratios and by optimizing the conditions of HSA depletion using anti-HSA IgY-microbeads in a “batch” mode. Affinity-purified anti-HSA IgY antibodies (3 mg/ml) were oxidized with sodium meta-periodate (5 mg/ml), at room temperature for 30 minutes, followed by dialysis against 4 L of Phosphate Buffered Saline (PBS), in a 2 ml/dialysis cassette (Pierce Product No. 66425: Slide-A-Lyzer Dialysis Cassettes, 10 k MWCO) at 4° C. for 1 h, with 3 changes of buffer. Oxidized IgY was incubated with Hydrazide microbeads (Pierce Product No. 53149) to obtain conjugation ratios of 5, 10, 15 and 20 mg IgY/ml microbeads. Conjugation was carried out at 4° C. overnight with rotation. After conjugation, microbeads were washed with 1M NaCl and followed by 3× with PBS, and stored as a 50% slurry in PBS.

### EXAMPLE 2

### Test of Depletion Efficiency of Anti-HSA IgY Microbeads Using Purified Human Serum Albumin

Titration of the binding efficiency of anti-HSA IgY microbeads were carried out using Handee Mini-Spin Column (Pierce Product No. 69705) and HSA-spiked PBS samples. Fifty microliters (50 μl) of 50% microbeads were centrifuged (8 seconds) in a spin column. Dried microbeads were quickly incubated with 25 μl samples containing 0.72, 1.39, 2.72, 7.35, 10.85 or 14.83 mg/ml HSA (Diagnostic Grade) (US Biological, Product No. A1327-15) in PBS measured by BCA protein assay. These represented total amounts of 18 μg, 35 μg, 68 μg, 184 μg, 271 μg and 371 μg protein, respectively. Binding reactions were performed in the column at room temperature for at least 30 minutes. IgY microbeads were gently resuspended once every 3-5 minutes using disposable pipette tips. After incubation, the column was inserted into an Eppendorf tube and centrifuged for 8 seconds at 14,000 rpm in a microfuge. Proteins in collected samples were quantified by BCA. Table 2 summarizes the experimental results, using different ratios of IgY microbead to target protein concentrations. These results were obtained with one-round of depletion, in most cases using quadruplicate samples.

TABLE 2
Efficiency of Anti-HSA IgY Microbeads for “Spiked” Samples in PBS

| | HSA Depletion Efficiency (%) | | | | | |
|---|---|---|---|---|---|---|
| IgY/Bead | 18 μg | 35 μg | 68 μg | 184 μg | 271 μg | 371 μg |
| (mg/ml) | (0.7 mg/ml) | (1.4 mg/ml) | (2.7 mg/ml) | (7.4 mg/ml) | (11 mg/ml) | (15 mg/ml) |
| 5 | 100 | 100 | 85 | 59 | n.t. | n.t. |
| 10 | 100 | 100 | 100 | 52 | 38 | 35 |
| 15 | n.t. | n.t. | n.t. | 61 | 43 | 47 |
| 20 | n.t. | n.t. | n.t. | 51 | 49 | 15 |

n.t.: not tested.

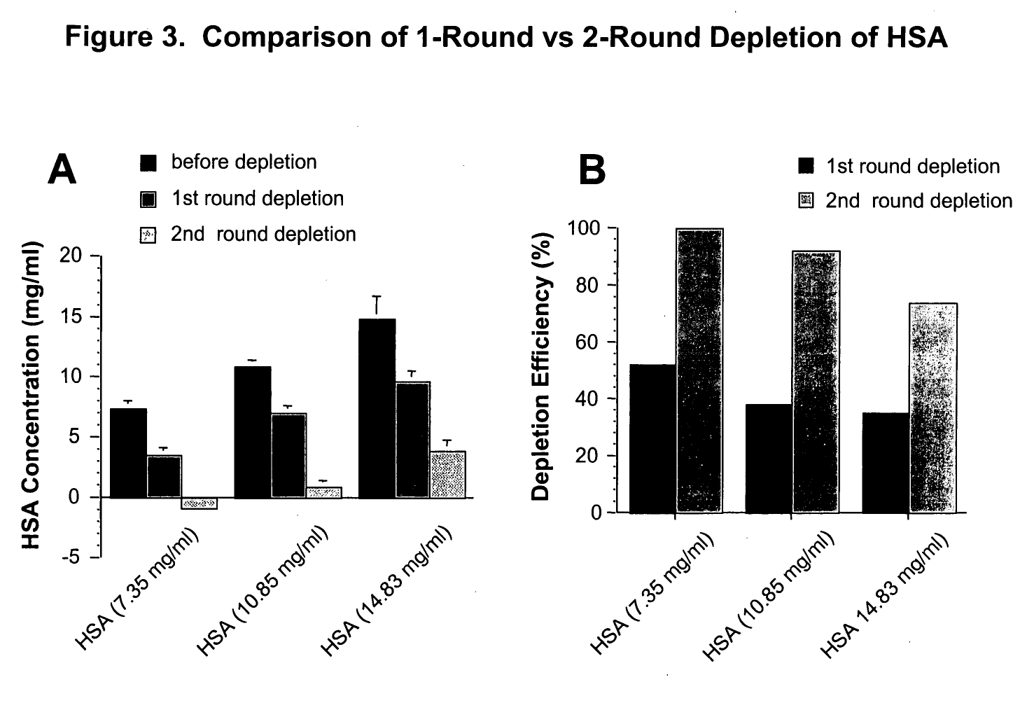

Titration was further carried out through a process of two serial rounds of depletion for removal of additional HSA. “10×” microbeads (=10 mg IgY/ml microbeads) were mixed with 25 μl of 7.35, 10.85 and 14.83 mg/ml HSA, equivalent to 184, 271 and 371 μg protein, respectively. Flow-through samples from 1^{st} round depletion were collected and subjected to a 2^{nd} round of depletion with the identical amount of fresh microbeads (FIG. 3).

### EXAMPLE 3

### Depletion of Human Serum Albumin (HSA) from Serum Samples using Anti-HSA IgY Microbeads

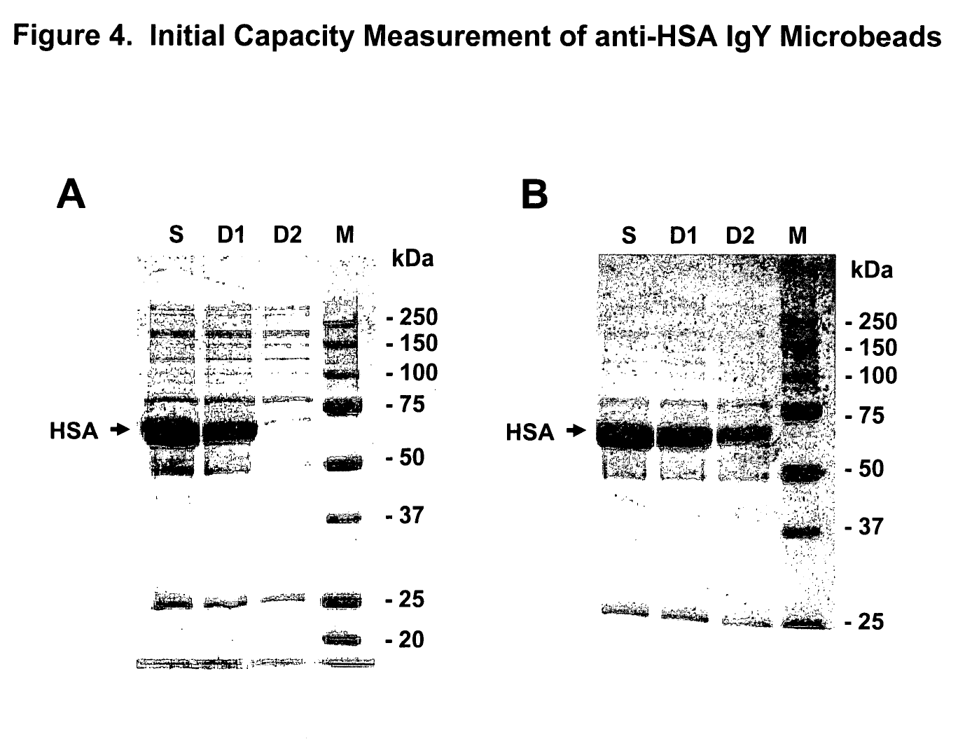

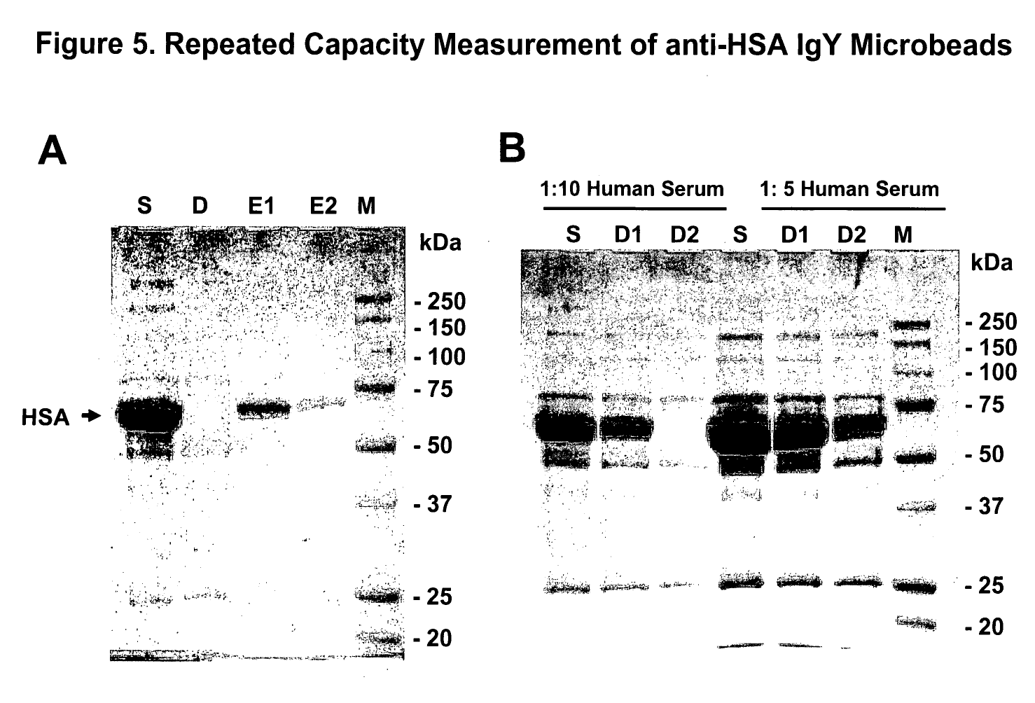

To test HSA depletion in a human (male) serum sample (Sigrna, H-1388, Lot 122K0424), either 4 μl or 10 μl human serum samples were diluted to a total of 25 μl in PBS. Two rounds of depletion were performed using “10×” microbeads (=10 mg IgY/ml microbeads) as described in Example 2. To analyze depletion results, 2 μl of collected sample were diluted to 201 μl in sample loading buffer and boiled for 3 min. After cooling, 15 μl (for 4 μl serum depletion) or 5 μl (for 10 μl serum depletion) samples were subjected to 10% SDS-PAGE, followed by Coomassie Blue R-250 staining (FIG. 4, A, 4 μl serum depletion; B, 10 μl serum depletion). Tests of depletion of HSA from human serum samples were repeated. Twenty-five microliter (25 μl) of diluted human serum (1:5 and 1:10 dilution in TBS) were subjected to 2 rounds of HSA depletion using 25 μl “5×” IgY microbeads (conjugation ratio: 5 mg IgY/ml microbeads). Results shown in FIG. 5, panel A was with 3 μl/lane of pooled materials from 4 experiments (serum dilution at 1:5). Lane D in panel A shows that all of the HSA was removed completely from the serum after 2 rounds of depletion. FIG. 5, panel B shows the results of each depletion from human serum diluted 1:5 or 1:10. As clearly depicted in the picture, HSA was completely removed by the 5× microbeads in 1:10 diluted serum, and about 90% depletion was observed when serum was diluted 1:5 (Panel B, Lanes D2). The microbead elution lanes (Panel A, lanes E1 and E2) show that HSA was the only protein removed from the serum. This elution fraction can be analyzed by proteomics techniques well known in the art, such as 2-dimensional gel electrophoresis and mass spectrometry, to sensitively analyze other proteins co-purifying with HSA. The use of two anti-HSA columns in series avoids the need for substantial sample dilution. Using this technique with 25 μl microbead (50 μl slurry) volumes in a batch mode, HSA was almost completely removed from 4 μl serum diluted 6-fold, and about 65% of the HSA was removed from 10 μl serum diluted 2.5-fold, in both cases without any noticeable loss of other proteins (data not shown).

### EXAMPLE 4

### Depletion of Human Immunoglobulin

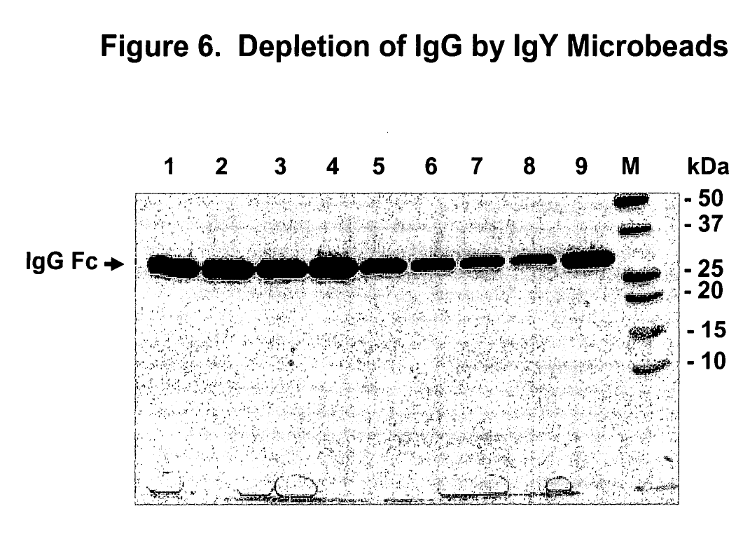

Affinity-purified anti-IgG-Fc IgY antibodies were covalently conjugated to UltraLink Hydrazide Microbeads using the method described in Example 1. Fifty microliter (50 μl) of purified human IgG-Fc (Calbiochem Catalog No. 401104) was spiked into PBS solution at concentrations of 10, 5, 2.5 or 1.25 mg/ml. In a control sample, human IgG-Fc (50 μl of 1.25 mg/ml, unoxidized) was spiked to PBS and incubated with unconjugated microbeads. The samples were subjected to one-round of depletion with anti-IgG-Fc-microbeads, by separation in a Handee Mini-Spin Column (Pierce Product No. 69705). The depleted samples were collected. Both starting materials (before depletion) and collected samples were diluted 10-fold (Lanes 1, 5), 5-fold (Lanes 2, 6), 2.5-fold (Lanes 3, 7) and 1.25-fold (Lanes 4, 8, and 9) to obtain a final concentration of 1 mg/ml, followed by SDS PAGE analysis. FIG. 6 shows that about 50% of IgG-Fc was depleted for the samples at a protein concentration of 5 mg/ml. 80% depletion was observed for the samples at 2.5 mg/ml and 1.25 mg/ml. The negative control unconjugated microbeads failed to bind to IgG-Fc (Lane 9).

### EXAMPLE 5

### Depletion of Human Apolipoprotein A-I

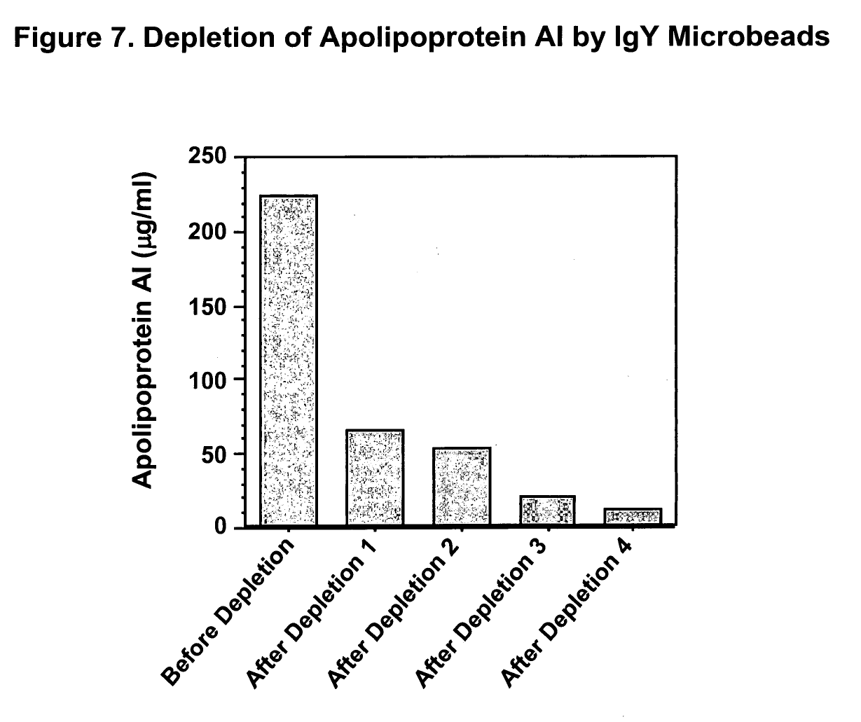

Affinity-purified anti-Apolipoprotein A-I IgY antibodies were covalently conjugated to CarboLink Agarose Beads (Pierce Biotechnology) essentially using the method described in Example 1. One hundred microliters (100 μl) of purified human Apolipoprotein A-I (Calbiochem Catalog No. 178452) was spiked into PBS solution at a final concentration of 0.225 mg/ml. The sample was sequentially subjected to four-rounds of depletion with anti-Apolipoprotein A-I-beads in a Handee Mini-Spin Column (Pierce Product No. 69705). The depleted samples from each round were collected, and subjected to BCA protein analysis. FIG. 7 shows about 95% of Apolipoprotein A-I was depleted after 4 rounds of depletion.

### EXAMPLE 6

### Capacity of IgY Composition in Immunoaffinity Separation of Abundant Proteins from Non-Abundant Proteins in Serum/Plasma Samples

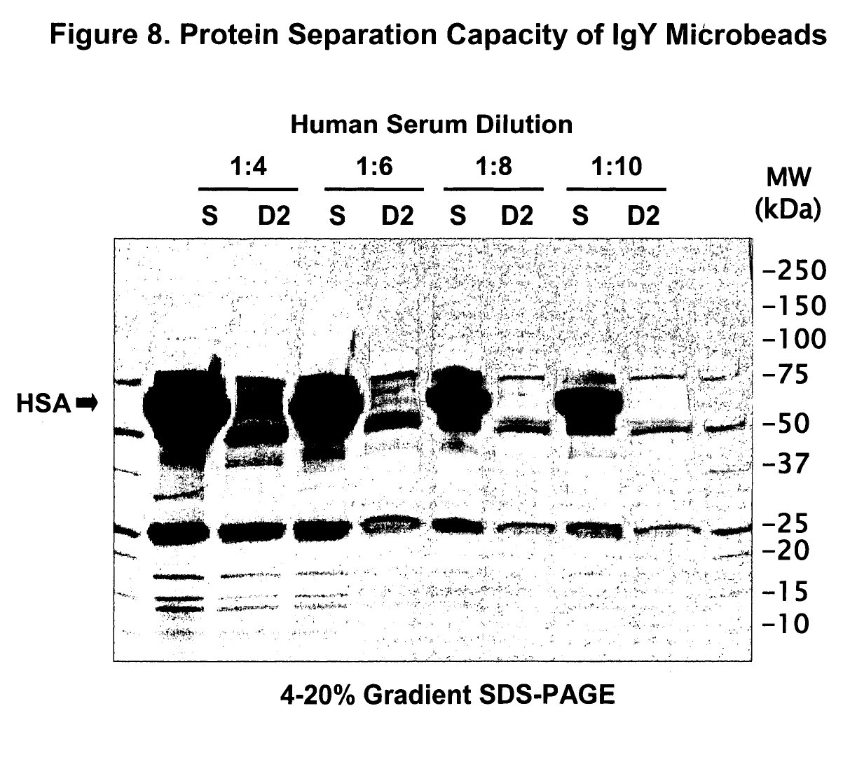

The binding capacity of IgY microbeads varies with different IgY antibodies against corresponding target proteins, and is related to the natural concentration of the target protein in serum/plasma, and to the avidity of the IgY antibody for its target, and to the concentration of capture antibody on the solid surface. To empirically test the capacity of anti-HSA IgY microbeads, human serum samples were diluted with TBS (Tris-Buffered Saline, 10 mM Tris-HCl, 0.15 M NaCl, pH 7.4) at ratios of 1:4, 1:6, 1:8, and 1:10. Equal volumes (25 μl) of the bed volume of microbeads and the diluted human serum samples (S) were mixed and incubated. The IgY microbeads were separated from the solution with a spin column device. The unbound materials (flow-through solution or 1^{st} fraction of depletion) were further mixed and incubated with another fresh 25 μl bed volume of IgY microbeads to repeat the separation process. This resulted in the 2^{nd} fraction of depletion (D2). The untreated diluted serum samples (S) and the 2^{nd} fraction of depletion (D2) were resolved on a 4-20% gradient SDS-PAGE under reducing conditions and were visualized via Coomassie Blue staining. As shown in FIG. 8, anti-HSA IgY microbeads completely depleted HSA from diluted serum in 2×25 μl batches if the serum is diluted greater than 4-fold. S: Starting, unfractionated human serum. D2: Unbound material after 2 rounds of anti-HSA depletion. Assuming 40 mg/ml HSA in undiluted serum, capacity is equivalent to ˜2.7 mg HSA captured/ml microbeads, where 1 ml microbeads contain about 10 mg IgY antibodies.

### EXAMPLE 7

### Specificity of IgY Composition in Immunoaffinity Separation of Abundant Proteins from Non-Abundant Proteins in Serum/Plasma Samples

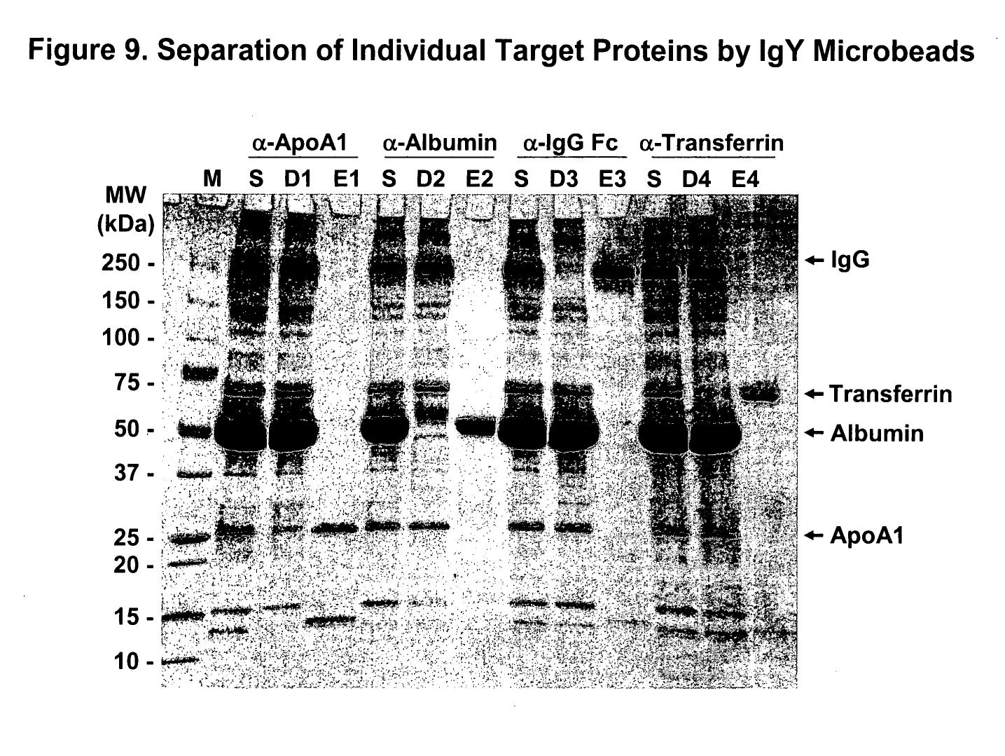

A critically important feature for any proteomics sample preparation composition or method is the specificity of capture of the target protein. Indeed, antibodies are among the most specific capture reagents available. To test the specificity of IgY microbeads for their intended targets, human serum samples were diluted in Tris-Buffered Saline (TBS dilution buffer, 10 mM Tris-HCl, 0.15 M NaCl, pH 7.4) based on abundance of the target protein, added to pre-packed IgY-microbead spin columns using empty Micro Bio-Spin Columns (Cat. No. 732-6204) and End-Caps (Cat. No. 731-1660) (Bio-Rad, Hercules, Calif., USA), and incubated at room temperature for 15 minutes with rotation. The samples depleted of the target proteins (flow-through) were collected in a 2 ml microcentrifuge tube by centrifugation (at 5,000×g for 15 seconds in a microcentrifuge). The spin columns were washed three times with TBS containing 0.05% Tween-20 (wash buffer) to remove residual unbound proteins, then the bound proteins were twice eluted with 0.1M Glycine, pH 2.5 (stripping buffer). For each elution, IgY microbeads in the spin column were mixed and incubated with the stripping buffer at room temperature for 3 minutes followed by centrifugation to collect the eluted proteins. After elution, the spin columns were immediately neutralized with 0.1 M Tris-HCl, pH 8.0, and pooled eluted fractions were neutralized with 1/10 volume 1 M Tris-HCl pH 8.0 (neutralizing buffer). Unfractionated samples, depleted fractions, and eluted-bound protein fractions were analyzed by 1-DE. A few representative examples are shown in FIG. 9. Albumin, IgG, and Transferrin are three highly abundant proteins in serum and plasma. Apolipoprotein A-I is the most abundant lipoprotein. Compared to unfractionated samples, Apolipoprotein A-I, Albumin, IgG, and Transferrin were effectively removed in flow-through samples (FIG. 9, lanes D1, D2, D3, and D4). Proteins bound to the corresponding IgY columns were predominantly the expected targets (FIG. 9, E1, E2, E3, and E4). Despite albumin being such a dominant protein in the serum, representing about half of the total protein mass, other proteins with far less abundance were effectively and specifically removed in the presence of albumin. These results demonstrate that IgY microbeads can efficiently and specifically separate complex serum proteins.

### EXAMPLE 8

### Efficiency of IgY Composition in Immunoaffinity Separation of Abundant Proteins from Non-Abundant Proteins in Serum/Plasma Samples

There is an unmet need to simultaneously remove multiple abundant plasma proteins. To determine the efficiency of simultaneous removal of several of the most abundant plasma proteins, individual IgY microbead compositions were mixed at an optimized ratio based on the relative abundance of their target proteins and avidity of IgY antibodies. Two types of mixed IgY microbeads, MIXED6 and MIXED12, were produced. The key features of two types of MIXED IgY microbeads are summarized in Table 3.

TABLE 3
MIXED IgY Microbead Products

| Products | MIXED6 | MIXED12 |
|---|---|---|
| IgY Antibody | Albumin | Albumin |
| Targets | IgG | IgG |
| | Transferrin | Transferrin |
| | Fibrinogen | Fibrinogen |
| | IgA | IgA |
| | IgM | IgM |
| | | α1-Antitrypsin |
| | | α2-Macroglobulin, |
| | | Haptoglobin, |
| | | Apolipoprotein A-I |
| | | Apolipoprotein A-II, |
| | | α1-Acid Glycoprotein |
| Spin Column | Two-step process | One-step process |
| Process | | |
| Separation | Remove about 88% total | Remove about 95% total |
| Efficiency | plasma proteins | plasma proteins |

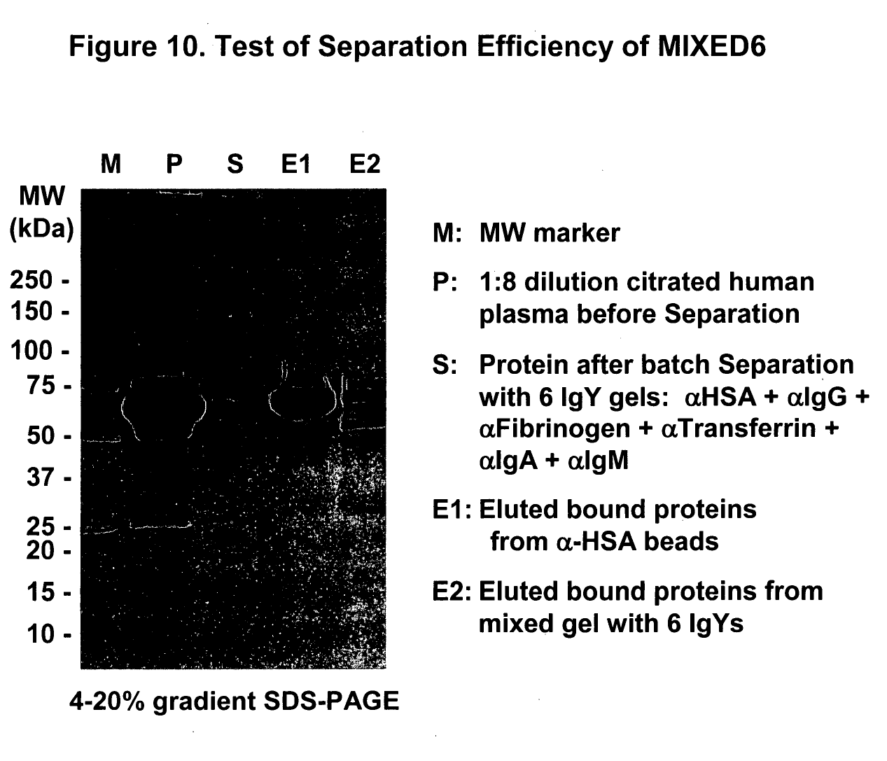

Human plasma samples were diluted and treated with MIXED6 IgY microbeads through a two-step spin column process, with the flow-through from the first anti-HSA antibody spin column then being passed through a spin column filled with the appropriate mixture of anti-HSA and 5 other microbead-coupled IgY antibodies. FIG. 10 shows the results of removal of six abundant plasma proteins using MIXED6. M, Molecular weight marker; P, 1:8 dilution citrated human plasma before depletion; D, P after depletion with MIXED6 IgY microbeads; E1, Eluted bound proteins from anti-HSA microbeads; E2, Eluted bound proteins from MIXED6 IgY microbeads. Proteins were visualized by 4-20% gradient SDS-PAGE with Coomassie Blue staining.

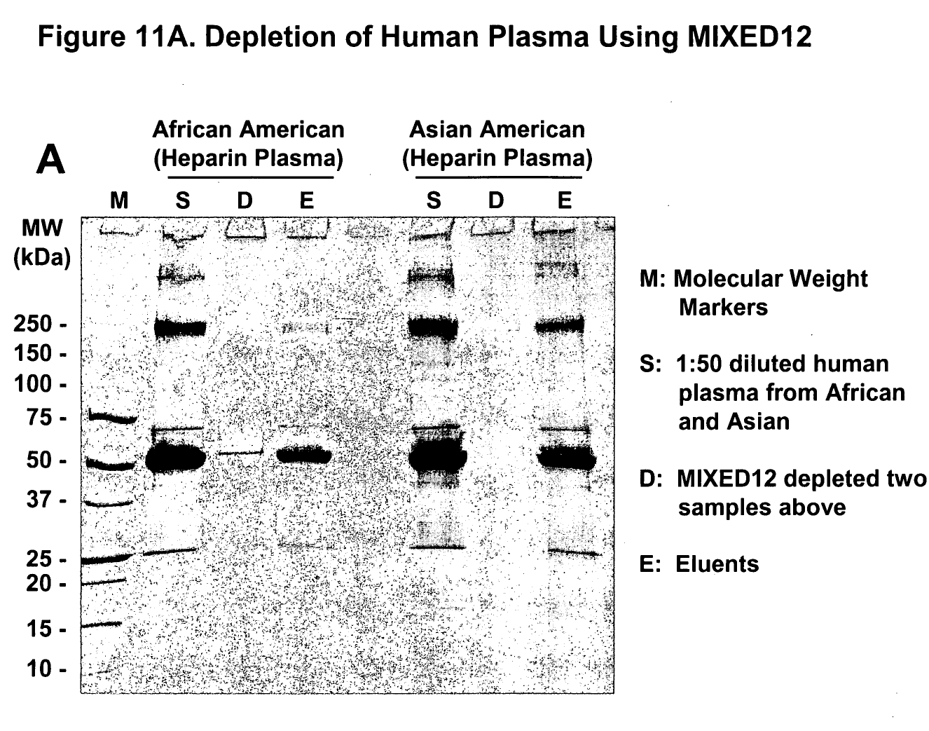

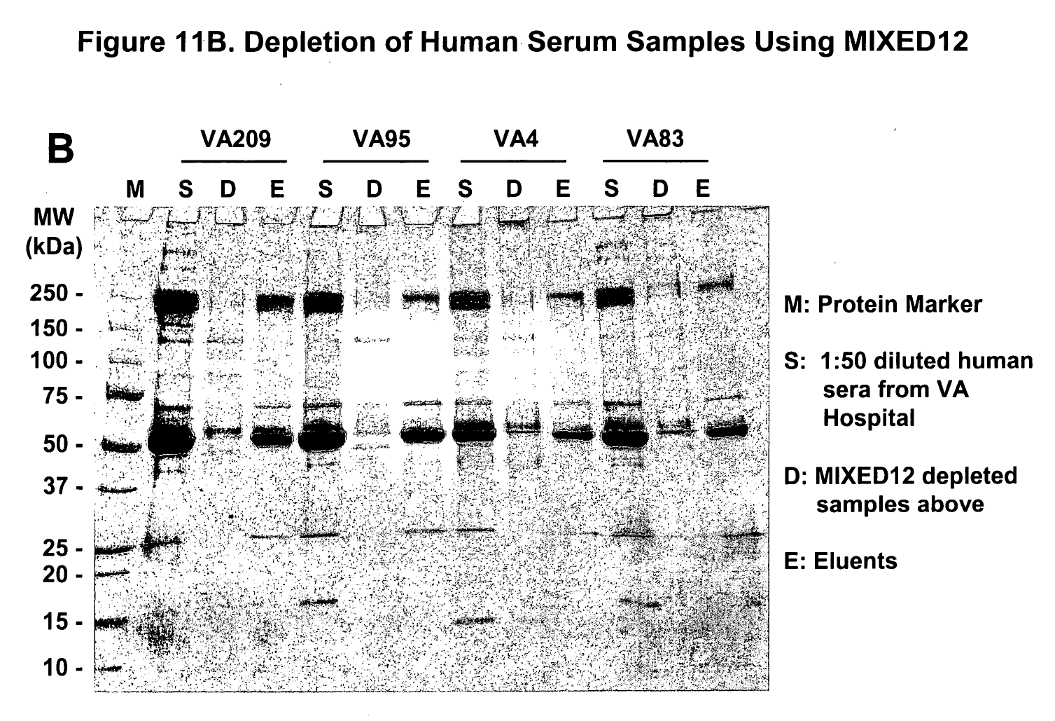

To enhance the convenience of use, a one-column system was employed for MIXED12. Plasma or serum samples were treated with MIXED12 spin columns essentially as described in Example 7. Two representative examples are shown in FIGS. 11A and 11B. Six sets of three fractions were loaded onto 4-20% SDS gel under non-reducing conditions: unfractionated samples before loading to spin columns (S), depleted of target proteins (D), and eluted-bound proteins (E). M is a molecular weight marker. Compared to unfractionated samples, the target proteins were effectively removed from the flow-through samples (Lanes D). Proteins bound to the corresponding IgY columns were mainly the expected targets (Lanes E). These results demonstrate that MIXED12 can efficiently and specifically separate complex serum proteins.

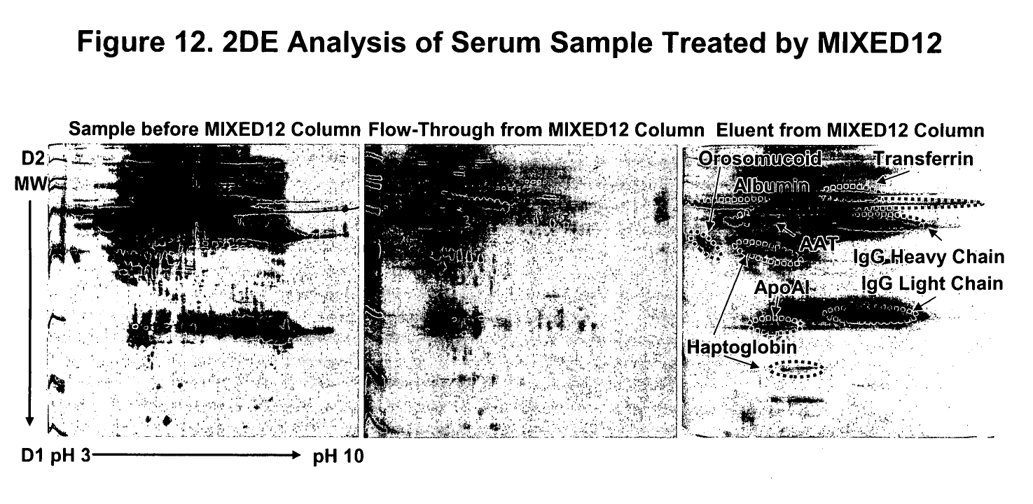

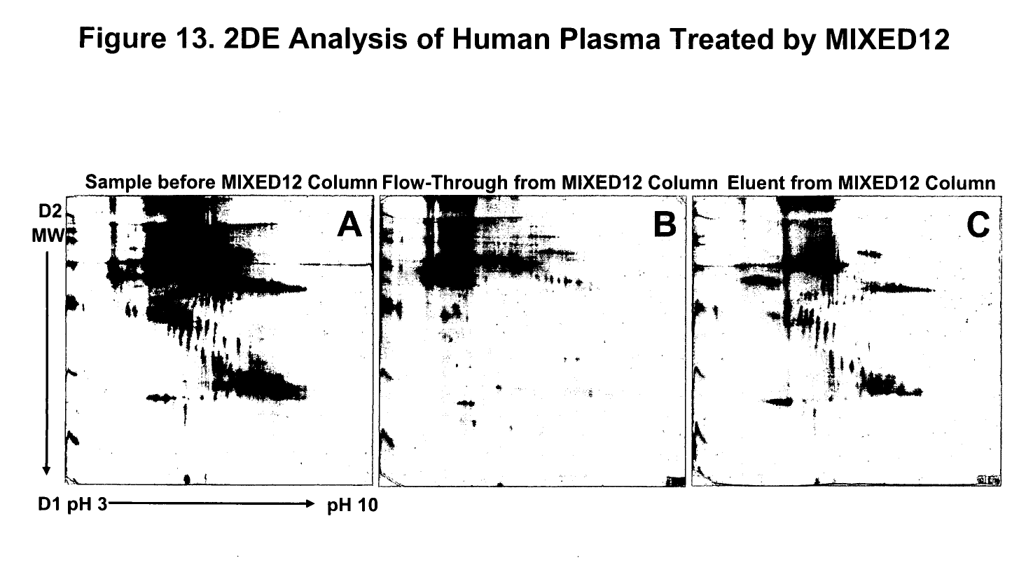

Plasma or serum samples treated with MIXED12 were further analyzed by two-dimensional gel electrophoresis (2DE). Three samples (unfractionated, depleted and eluted samples, ˜100 μg each) were precipitated using acetone and dissolved in a rehydration solution (9.5 M urea, 4% CHAPS, 18 mM DTT, 0.5% IPG buffer pH 3-10, trace of bromophenol blue), and loaded into a 13 cm long pH3-10 NL Immobiline™ DryStrip (Amersham Biosciences). The DryStrip was laid on the sample solution, covered with paraffin oil and allowed to rehydrate overnight. IPGphor (Amersham Biosciences) was used for the first dimension IEF, for a total running time of 42,000 Vh. Prior to the second dimension separation, the strip was equilibrated (50 mM Tris-HCl pH 8.8, 6 M urea, 30% glycerol, 2% SDS, trace of bromophenol blue, 100 mg of DTT was added per 10 mL solution prior to use) for 15 minutes in a screw-cap culture tube followed by alkylation with Iodoacetamide (25 mg/ml) for 15 minutes. Proteins were separated vertically by second-dimensional SDS-PAGE (12% acrylamide) at 10° C. and visualized by staining with Gel Code™ (Pierce Chemical Co.). The resulting 2DE images were analyzed by comparisons with standard human serum and plasma 2DE maps found in the pubic domain FIGS. 12 and 13. In comparison, many protein spots previously obliterated by abundant proteins were revealed in the flow-through, depleted fraction. Selective removal of highly-abundant proteins significantly improved the 2DE resolution of plasma proteins. The removed proteins were detected. Definitive protein identification can be carried out after cutting out the gel spots, followed with trypsin digestion and peptide mass fingerprinting using MALDI-TOF mass spectrometry analysis.

### EXAMPLE 9

### Recyclability of IgY Composition and Reproducibility of Immunoaffinity Separation

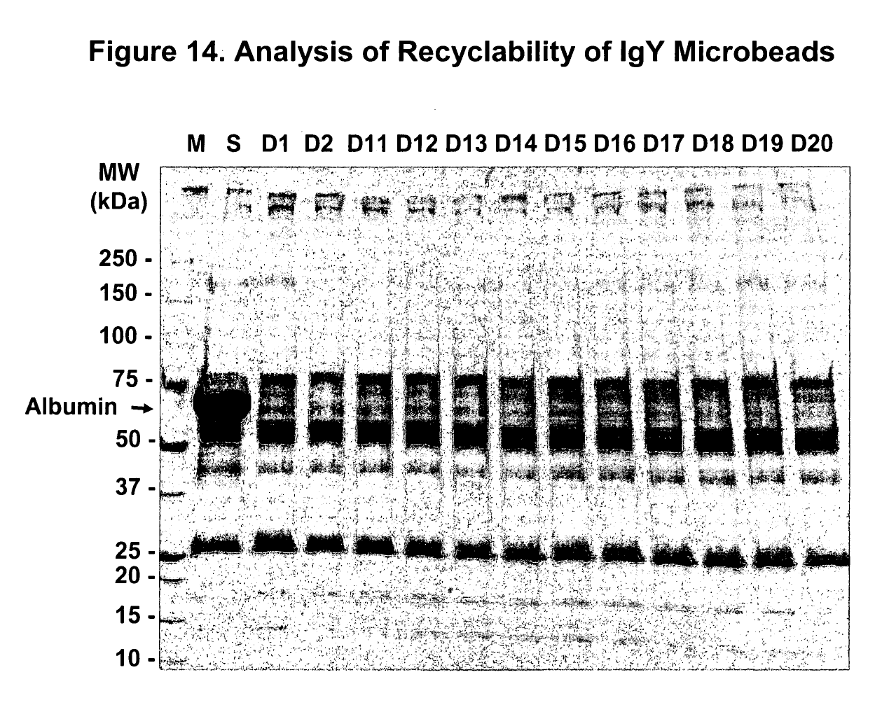

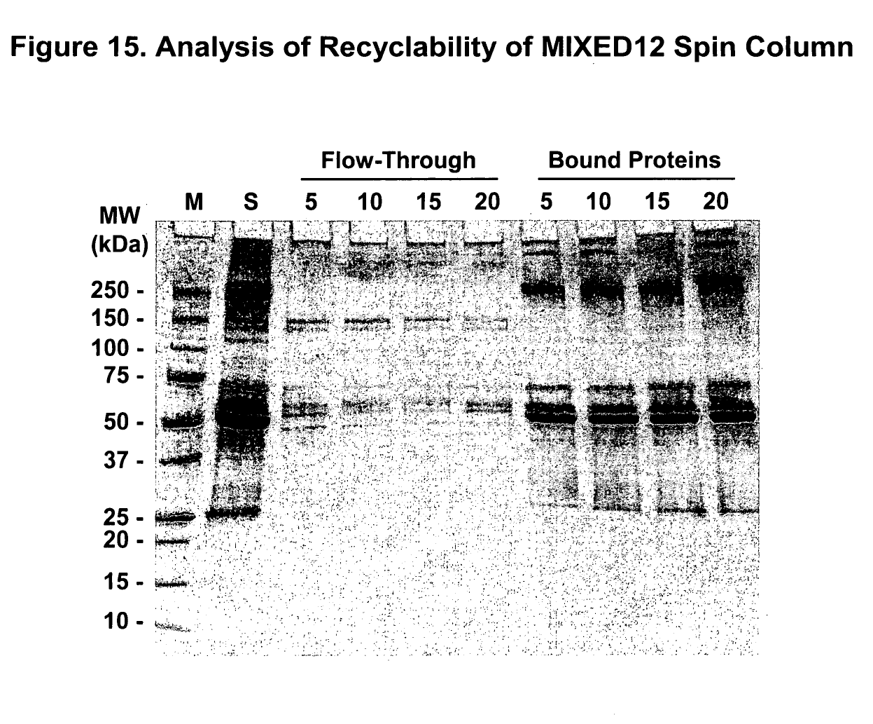

Reproducibility measures the accuracy that a product can perform through a repetitive process. Recyclability is an indication of the endurance of a product and its capability of being regenerated without loss of either capacity or specificity. To evaluate the reusability of the IgY microbead columns of the present invention, an important factor in their economic use, the depletion efficiency of IgY microbead columns over multiple cycles was systematically analyzed. First, anti-HSA IgY microbead spin column was used to separate proteins from aliquots of the same human serum sample 20 times in succession. FIG. 14 shows the selected samples analyzed by IDE 4-20% SDS PAGE under non-reducing conditions. M, Molecular weight marker; S, Sample of human serum sample; D1, D2, D11-20, Samples of flow-through from the corresponding cycle of the same anti-HSA IgY spin column. The fractions depleted of albumin from D1 to D20 are virtually identical, demonstrating high reproducibility and recyclability of anti-HSA IgY microbeads. MIXED12 product was also tested for its recyclability and reproducibility. In order to make the MIXED12 column reusable, bound proteins must be efficiently and completely removed without damaging the antibodies coupled to the column. In addition, all 12 targeted proteins must be eluted from the column under the same buffer conditions. First, individual IgY microbead columns with single protein targets were tested with serum or plasma samples running through multiple cycles. ELISA or Western blotting methods were used to evaluate the depletion efficiency by assaying residual proteins in the depleted fractions. Under identical binding, washing, stripping, neutralizing and reequilibrating buffer conditions, all 12 proteins were efficiently removed from their corresponding columns. Twenty aliquots of a human serum sample were sequentially run through cycles in the same MIXED12 spin column containing the same microbead composition. The flow-through and eluted fractions were collected. Selected fractions were analyzed by 1DE, ELISA, and Western Blotting. As illustrated in FIG. 15, indistinguishable protein banding patterns were observed in samples collected at cycles 5, 10, 15, and 20, indicating high reproducibility with a single column over multiple cycles. The ELISA and Western blotting results for cycle #20 are summarized in Table 4. Among the twelve proteins, Albumin, IgG, IgA, Transferrin, α2-Macroglobulin, Apolipoprotein AI and AI, and Fibrinogen were reproducibly removed to near completion. Haptoglobin, α1-Antitrypsin, Orosomucoid, and IgM were also significantly removed, although with slightly less efficiency than the other eight proteins.

TABLE 4
Depletion Efficiency of MIXED12 after Multiple Cycles

| | Relative abundance in | | Method of |
|---|---|---|---|
| Protein | serum (average %)^{a} | % Removal | Detection |
| Albumin | 54 | >99.5% | ELISA |
| Immunoglobulin G | 17 | >99.5% | WB |
| Transferrin | 3.3 | >99.5% | WB |
| Haptoglobin | 3.0 | 92-95% | WB |
| α1-Antitrypsin | 3.8 | >95.0% | WB |
| α2-Macroglobulin | 3.6 | >99.5% | WB |
| Immunoglobulin A | 3.5 | >99.5% | WB |
| Immunoglobulin M | 2.0 | 90-95% | ELISA |
| Orosomucoid | 1.3 | 92-95% | WB |
| Apolipoprotein AI | 3.0 | >99.5% | WB |
| Apolipoprotein AII | 1.0 | >99.5% | WB |
| Fibrinogen | 3.0 (plasma)^{b} | >99.5%* | WB |
| ^{a} and ^{b}Approximate weight-based protein abundance value in normal serum [Putnam, F. R. 1984. The Plasma Proteins, vol. IV, Academic Press, Orlando, FL. and Tybjaerg-Hansen, A. B. et al. 1997. A common mutation (G-455--> A) in the beta-fibrinogen promoter is an independent | | | |
| # predictor of plasma fibrinogen, but not of ischemic heart disease. A study of 9,127 individuals based on the Copenhagen City Heart Study. J Clin Invest. 99: 3034-3039]. | | | |

*The data for Fibrinogen were obtained in a separate experiment using an individual antibody spin column and human plasma sample.

### EXAMPLE 10

### Effectiveness of Anti-Human Protein IgY Composition in Immunoaffinity Separation of Orthologous Proteins from Plasma Samples of Other Mammals

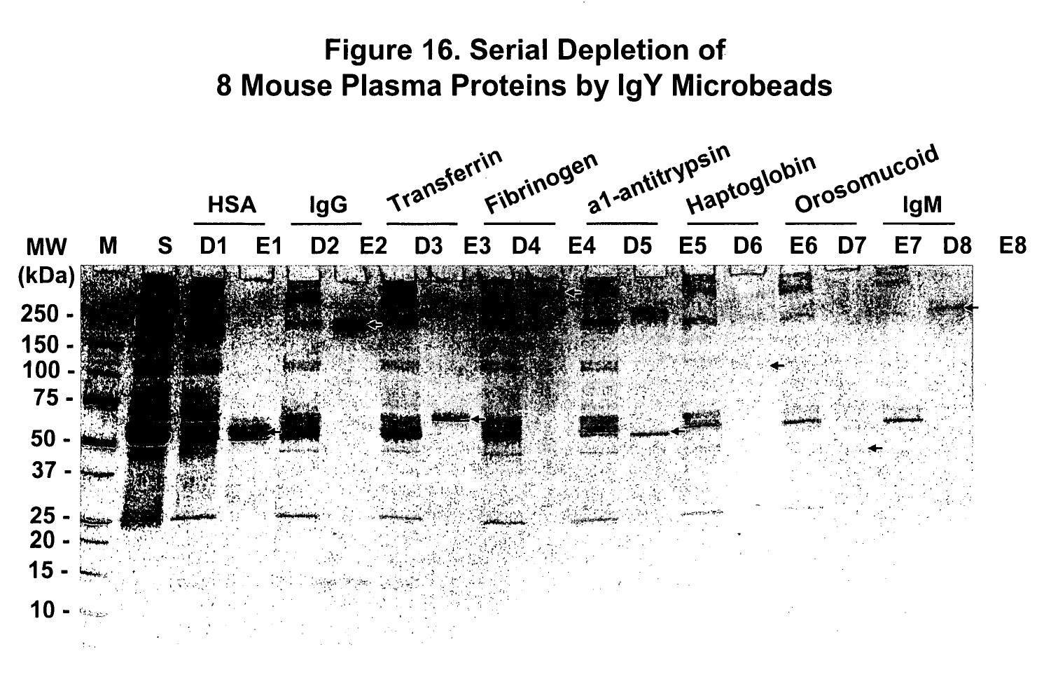

Due to the sequence similarity of many serum/plasma proteins between human and rodents, and the great evolutionary distance between birds and mammals, chicken antibodies against human proteins are likely to cross-react with their rodent orthologs. Mouse plasma samples were tested individually with several of the present IgY antibody microbead compositions against human plasma proteins in Western blot assays. Eight anti-human protein IgY antibody microbead compositions bound their corresponding mouse plasma proteins. To further confirm the Western blotting results, mouse plasma was sequentially run through eight IgY microbead columns, each with a different antibody. Collected fractions were analyzed by 1-dimensional SDS-PAGE (1DE) (FIG. 16). Specific protein depletion was clearly revealed, demonstrating that IgY antibodies directed against these human proteins, except Orosomucoid, can effectively be used to separate orthologous mouse plasma proteins. Removal of Orosomucoid was not detected by SDS-PAGE despite the fact that antibody cross-reactivity to the same protein of mouse and rat origin was confirmed by Western blot assay. In FIG. 16, M, Molecular weight marker; S, Unfractionated mouse plasma; D1, Plasma depleted of albumin; E1, Eluted-bound protein to anti-HSA IgY microbeads; D2, D1 depleted of IgG; E2, Eluted-bound protein to anti-IgG IgY microbeads; D3, D2 depleted of Transferrin; E3, Eluted-bound protein to anti-Transferrin IgY microbeads; D4, D3 depleted of Fibrinogen; E4, Eluted-bound protein to anti-Fibrinogen IgY microbeads; D5, D4 depleted of α1-antitrypsin; E5, Eluted-bound protein to anti-α1-Antitrypsin IgY microbeads; D6, D5 depleted of Haptoglobin; E6, Eluted-bound protein to anti-Haptoglobin IgY microbeads; D7, D6 depleted Orosomucoid; E7, Eluted-bound protein to anti-Orosomucoid IgY microbeads; D8, D7 depleted of IgM; E8, Eluted-bound protein to anti-IgM IgY microbeads. Arrows indicate the target proteins.

### EXAMPLE 11

### Comparison Binding Specificity of Anti-HSA IgY Composition to Anti-BSA IgY Composition

As demonstrated in Example 10, at least seven IgY microbead compositions directed against human plasma proteins can effectively bind to orthologous mouse proteins. In addition, anti-HSA IgY cross-reacts to albumin in several different species, such as mouse, rat, pig, and goat. Bovine serum albumin (BSA) is also an abundant protein present in large amounts in many tissue culture media. To assess whether anti-BSA IgY has same binding capacity and cross-species host range as anti-HSA IgY, a comparison experiment was performed. Human, cow, mouse, rat, pig, goat and dog serum samples were diluted 1:20 in TBS. Hundred microliters (100 μl) of anti-HSA or anti-BSA IgY microbeads were mixed with 100 μl of each diluted serum sample in a spin column. After 15 minutes of incubation with rotation, the albumin-depleted fraction was removed by brief centrifugation. The beads were then washed three times with TBS. The bound albumin was eluted with stripping buffer (0.1M Glycine-HCl, pH 2.5). The eluted fraction was neutralized immediately with 1 M Tris-HCl buffer pH 8.0. Protein concentration was measured by the BCA method following supplier's instruction (Pierce). Table 5 shows the comparison of the binding capacity of anti-HSA IgY microbeads and anti-BSA IgY microbeads to albumins of other species in duplicated experiments.

TABLE 5
Depletion Efficiency of Anti-HSA and BSA IgY Microbeads

| | |
|---|---|
| Anti-HSA IgY | Anti-BSA IgY |

mg antigen bound to the microbeads

| Species | Test 1 | Test 2 | Test 1 | Test 2 |
|---|---|---|---|---|
| Human | 2.00 | 2.22 | 1.60 | 1.72 |
| Bovine | 1.12 | 1.12 | 1.38 | 1.45 |
| Mouse | 1.46 | 1.51 | 1.10 | 1.46 |
| Rat | 1.22 | 1.18 | 0.88 | 0.93 |
| Pig | 1.28 | 1.22 | 1.38 | 1.51 |
| Goat | 1.20 | 1.12 | 1.77 | 1.98 |
| Dog | 1.06 | 1.06 | 0.95 | 1.09 |

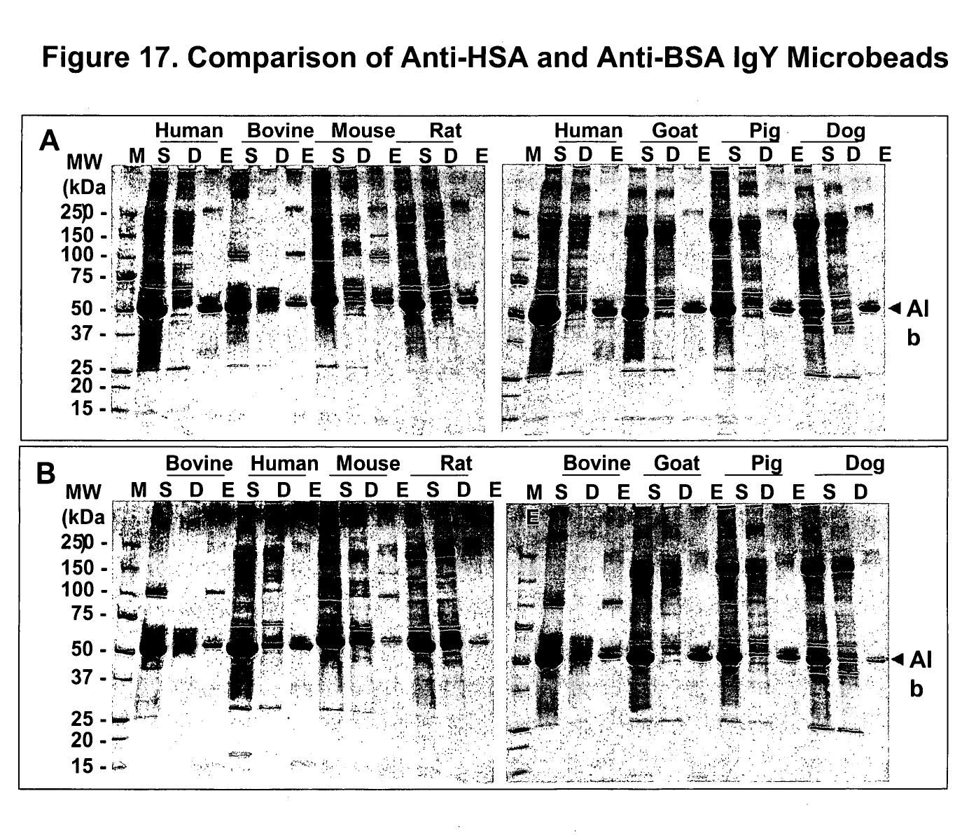

As shown in the table, anti-HSA IgY and anti-BSA IgY displayed quite distinct binding patterns to serum albumins of other mammalian species. Anti-HSA IgY has higher cross-reactivity to mouse and rat albumin, while anti-BSA IgY binds more goat and pig albumin. These different patterns were further illustrated in FIG. 17. Unfractionated, depleted and eluted fractions of each serum sample from Example 11, Test 1 were analyzed on 1D SDS-PAGE. After treatment with anti-HSA IgY microbeads, albumin in human, mouse, rat, pig, and dog sera was completely or almost completely removed (Panel A, lanes D under corresponding species names). In contrast, anti-BSA IgY beads efficiently removed albumin only from bovine, goat, and pig sera. The majority of mouse and rat albumin was not captured by the anti-BSA IgY microbeads; these rodent albumins still remained in the flow-through fractions (Panel B, lanes D under corresponding species names). This finding is consistent with the differences noted in the quantitative binding study above, and confirms significant differences in cross-species albumin reactivity between anti-HSA IgY microbeads and anti-BSA IgY microbeads.

### EXAMPLE 12

### Indirect Linkage of IgY Antibodies to Solid Support via Alternative Affinity Binding Reagents: Biotin and Avidin or Streptavidin

Covalent coupling of IgY antibodies to solid support via a bifunctional hydrazide linkage is shown as an example for indirect linkage, and alternative strategy for coupling antigen affinity purified IgY antibodies to solid supports such as microbeads, nanoparticles, etc. Mild oxidation of IgY with sodium periodate will produce reactive aldehydes on the carbohydrate moieties of the Fc portion that then can be alkylated by hydrazides. This approach is advantageous for antibodies because they become covalently modified in a manner that maintains immunological reactivity, and it is ideal for polyclonal IgY antibodies because they are heavily glycosylated. The configurations of this chemistry are quite flexible and encompassed by the claims in this application. In addition to the method described in Example 1, where carbohydrates on the Fc portion of antigen affinity purified IgY oxidized by sodium metaperiodate were covalently linked to a hydrazide-coated microbead surface, other configurations are envisioned. For example, periodate-oxidized IgY is reacted with a bifunctional linker molecule containing a hydrazide at one end and a ligand at the other end. The resulting IgY-ligand molecule then binds tightly and specifically with a ligand receptor bound to a solid surface, such as a microbead.

Two specific examples are illustrated below (biotin/avidin and biotin/streptavidin). In these examples, the bifunctional linker molecule comprises hydrazide at one end and the ligand is biotin at the other end. Coupling of the biotinylated IgY to a solid surface is mediated through avidin or streptavidin. Coupling of Biotin-Hydrazide to IgY antibody is done according to the manufacturer's instructions: Pierce Biotechnologies (Product number 21340: EZ-Link™ Biotin Hydrazide, Spacer Arm: 15.7 Å, Molecular Weight: 258.34; or Product Number 21340 EZ-Link™ Biotin-LC-Hydrazide, Spacer Arm: 24.7 Å, Molecular Weight: 371.50).

After biotinylating the carbohydrates on the IgY antibodies, the molecules are reacted with a planar surface, microbeads or nanobeads coated with avidin or streptavidin. Examples of such solid support products include Dynabeads MyOne™ Streptavidin, Dynabeads® M-280 Streptavidin or Dynabeads® M-270 Streptavidin from Dynal Biotech (Brown Deer, Wis.), or Power-Bind™ Streptavidin Microparticles from Seradyn (Indianapolis, Ind.).

### EXAMPLE 13

### Multiplex (96-Well Plate) Format of Application

IgY microbeads and IgY composition can also be applied to multi-well or array format apparatus, such as microplates having a membrane at the bottom of each well. Two hundred microliter (200 μL) of a slurry (50%) containing the MIXED12 IgY microbeads is aliquoted into a 96-well filter plate (Cat # F20036 or F20009 from Innovative Microplate, MA, USA). To remove the buffer, the plate is centrifuged at 1,000 rpm for 1 minute in an Eppendorf bench top centrifuge with plate adapter. The separation process includes the following steps: Rinse/centrifuge the plate 2-3 times with 100 μL PBS. Discard the PBS rinse. Dilute 1 μL plasma in 99 μL of PBS, add to well, mix with pipette tip and incubate for 30 min at room temperature on a shaker. Centrifuge the plate as described above and collect Fraction 1 (˜100 μL) in NUNC cat # 260251. Add 100 μL PBS/0.02% Tween-20 to the well. Centrifuge and collect Fraction 2 into fresh NUNC cat #260251. Add 100 μL 0.1 M glycine pH 2.5 to the well, centrifuge and collect Fraction 3. Add 100 μL 0.1 M Tris pH 8.0 to the well, centrifuge and add to fraction 3 (˜200 mL). The resulting Fraction 1, Fraction 2 and Fraction 3 are then analyzed using standard analytical techniques such as, for example, an H50 chip surface on a SELDI mass spectrometer (Ciphergen Biosystems, Freemont, Calif.). Note that H50 is a selective surface and does not capture all protein in a sample, but a subset. The multi-well and array format can also be further expanded to higher density (384-well, 1536-well formats) microplates. In addition, the IgY compositions can also be used in microfluidics instruments, such as the LabChip 90 Electrophoresis System or the LabChip 3000 Drug Discovery System (Caliper Life Sciences, Hopkinton, Mass.).

### EXAMPLE 14

### Mixing IgY Antibodies at Certain Ratio before Covalently Linked to Solid Support

Individual IgY antibodies can be conjugated to solid supports to form individual IgY compositions. In addition, a group of different IgY antibodies can also be simultaneously linked to a solid support matrix. The groups of IgY antibodies can be mixed in certain ratios for optimized immunoaffinity separation of target proteins. One example is to conjugate the 12 IgY antibodies used in MIXED12 through a process that is different from that of Example 8. The 12 IgY antibodies against HSA, IgG, Fibrinogen, Transferrin, IgA, α2-Macroglobulin, IgM, α1-Antitrypsin, Haptoglobin, Apolipoprotein A-I, Apolipoprotein A-II, and α1-Acid Glycoprotein are first mixed in a ratio based on the relative abundance of these 12 proteins in serum/plasma and the capacity of individual IgY microbeads. The mixed population of antibodies is then oxidized with sodium meta-periodate (5 mg/ml) at room temperature for 30 minutes, followed by dialysis against Phosphate Buffered Saline (PBS) to remove residual oxidant. Oxidized IgY antibodies are incubated with UltraLink® Hydrazide beads (Pierce Product No. 53149) to obtain conjugation ratios of 10 to 15 mg IgY/ml beads. Conjugation is carried out at 4° C. overnight with rotation. After conjugation, the IgY-coupled microbeads are thoroughly washed with 1M NaCl, followed by Tris-Buffered Saline (TBS, 10 mM Tris-HCl, 0.15 M NaCl, pH 7.4), and stored as a 50% slurry in TBS with 0.01% NaN₃ at 4° C.

All patents, patent applications, provisional applications, and publications referred to or cited herein are incorporated by reference in their entirety to the extent they are not inconsistent with the explicit teachings of this specification.

1. An affinity separation composition for separating one or more targets in a complex mixture comprising one or more affinity reagents linked to a solid support and oriented in a manner to facilitate the activity of the affinity reagents wherein said affinity reagents are capable of binding specific targets by affinity recognition and said solid support is capable of mediating separation of the affinity reagent-target complex from the mixture containing non-specific targets.

2. The affinity separation composition of claim 1 wherein the affinity reagents are IgY polyclonal antibodies having an Fc region and Fab regions, proteins, recombinant proteins, peptides, nucleotides, polymers or a mixture thereof and the target is an antigen.

3. The affinity separation composition of claim 2 wherein the affinity reagents are IgY antibodies, having an Fc region and Fab regions, that are covalently linked to the solid support with a bond to oxidized glycosylation moieties in the Fc region of the polyclonal IgY antibodies wherein said polyclonal IgY antibodies are made by immunizing and boosting a bird with an antigen and said antibodies specifically bind with said antigen through the Fab regions.

4. The affinity separation composition of claim 3 wherein the bird is a chicken.

5. The affinity separation composition of claim 3 wherein the solid support contains hydrazide groups that form a hydrazone bond with the oxidized glycosylation moieties in the Fc region of the polyclonal IgY antibodies.

6. The affinity separation composition of claim 3 further comprising one or more antigens present in the complex mixture wherein said antigens are affinity recognized and bound by the Fab regions of the IgY antibodies.

7. The affinity separation composition of claim 3 wherein the antigen is a protein, a peptide, a protein-protein complex, a protein-nucleotide complex, a protein-sugar/lipid complex, a biological complex, a nucleotide, a cell or a subcellular organelle, a microorganism, all of which can induce antibodies in the bird.

8. The affinity separation composition of claim 3 wherein the target is a protein.

9. The affinity separation composition of claim 8 wherein the target is a human protein.

10. The affinity separation composition of claim 8 wherein the human protein used to immunize the bird is Albumin, IgG, Fibrinogen, Transferrin, IgA, α2-Macroglobulin, IgM, α1-Antitrypsin, Haptoglobin, α1-Acid Glycoprotein, Apolipoprotein A-I and Apolipoprotein A-II or High-Density Lipoprotein, or mixtures thereof.

11. The affinity separation composition of claim 3 further comprising a human protein specifically bound to the antibody wherein the human protein is Albumin, IgG, Fibrinogen, Transferrin, IgA, α2-Macroglobulin, IgM, α1-Antitrypsin, Haptoglobin, α1-Acid Glycoprotein, Apolipoprotein A-I and Apolipoprotein A-II or High-Density Lipoprotein, or mixtures thereof.

12. The affinity separation composition of claim 3 wherein the support is covalently linked with one or more populations of polyclonal IgY antibodies wherein each population of polyclonal antibodies bind with a different human protein.

13. The affinity separation composition of claim 12 that contains 2 or more populations of antibodies covalently linked to the solid support wherein said populations of antibodies are (a) independently linked to a separate solid support first and then mixed in a given ratio for an effective affinity separation composition or (b) mixed in an effective affinity-separation ratio first and then linked to the solid support.

14. The affinity separation composition of claim 13 that contains at least 6 different populations of polyclonal IgY antibodies.

15. The affinity separation composition of claim 14 that contains 6 populations of IgY antibodies that specifically bind with Albumin, IgG, Fibrinogen, Transferrin, IgA and IgM.

16. The affinity separation composition of claim 13 that contains at least 12 different populations of polyclonal IgY antibodies.

17. The affinity separation composition of claim 16 that contains 12 populations of IgY antibodies that specifically bind with at least 12 proteins selected from the group consisting of Albumin, IgG, Fibrinogen, Transferrin, IgA, α2-Macroglobulin, IgM, α1-Antitrypsin, Haptoglobin, α1-Acid Glycoprotein, Apolipoprotein A-I and Apolipoprotein A-II or High Density Lipoprotein.

18. The affinity separation composition of claim 1 wherein said solid support comprises:
a. a defined shape to orient the affinity reagent to facilitate binding of the affinity reagent with the target;
b. a surface material for linking the solid support to the affinity reagent; and
c. a core material for mediating separation of the target-bound affinity reagent from the complex mixture.

19. The affinity separation composition of claim 18 wherein said defined shape is a sphere or an area surface.

20. The affinity separation composition of claim 19 wherein said sphere is a microsphere or a nanosphere and the area surface are wells or channels that can contain solutions or allow solutions to flow.

21. The affinity separation composition of claim 19 wherein said sphere is in multiplex format of a mixture of spheres and the wells are microplate wells in a format of 96 wells, 384 wells, or 1536 wells per plate.

22. The affinity separation composition of claim 18 wherein said surface material is a chemical or biological group that is capable of linking the solid support to the affinity reagent.

23. The affinity separation composition of claim 22 wherein the chemical or biological groups link the solid support to the affinity reagent (a) directly and covalently or (b) indirectly and non-covalently by a chain of specific ligand-interactions or (c) a combination of (a) and (b).

24. The affinity separation composition of claim 23 wherein the chemical group is a hydrazide.

25. The affinity separation composition of claim 23 wherein the biological group is the combination of biotin and avidin, or biotin and streptavidin.

26. The affinity separation composition of claim 18 wherein said core material is an acrylamide/azlactone copolymer, a polystyrenedivinylbenzene, a polystyrene, an agarose, a polymer, a resin, a polyester, a metal, a paramagnetic material, a magnetic material or mixtures thereof.

27. The affinity separation composition of claim 26 wherein the core material is coated with a surface material and in defined shapes is a sphere or an area surface.

28. The affinity separation composition of claim 27 wherein the core material is coated with a hydrazide.

29. The affinity separation composition of claim 27 wherein the defined shape is a microsphere or a nanosphere.

30. The affinity separation composition of claim 18 wherein said solid support is placed in a device to mediate separation of affinity reagent-target complex from the complex mixture.

31. The affinity separation composition of claim 30 comprising a chromatographic column, a multiple-well plate, or a microfluidic apparatus.

32. The affinity separation composition of claim 31 wherein the column is a spin column, a conventional liquid chromatographical column, an FPLC or HPLC column, or a combination of them operable through a manual or automated process.

33. The affinity separation composition of claim 31 wherein the multiple well plate is a plate or micro-plate containing 8-wells, 16-wells, 64-wells, 96-wells, 384-wells, or 1536-wells per plate.

34. A polyclonal IgY antibody composition which comprises:
a. a solid support containing hydrazide moieties;
b. polyclonal IgY antibodies having an Fc region and Fab regions wherein the antibodies are covalently linked to said support with a hydrazone bond to oxidized glycosylation moieties in the Fc region; and
c. an antigen specifically bound to Fab regions of said IgY antibodies wherein said polyclonal IgY antibody is made by immunizing a bird with the antigen.

35. The polyclonal IgY composition of claim 34 wherein the antibody is anti-HSA IgY antibody and the antigen is HSA.

36. The polyclonal IgY composition of claim 34 wherein the solid support is a microbead or a nanobead.

37. A method of affinity separating at least one target protein in a complex human protein mixture which comprises:
a. providing a complex human protein mixture which contains at least one target protein,
b. contacting the complex protein mixture with a polyclonal IgY composition of claim 3 wherein at least one target protein in the complex mixture specifically binds with the polyclonal IgY antibodies in the Fab regions and
c. recovering the treated complex protein mixture wherein the concentration of at least one target protein has been substantially reduced.

38. The method of claim 37 wherein the complex human protein mixture is plasma, serum, derived from tissue, cerebrospinal fluid, bronchial alveolar lavage, vitreous humor, nipple aspirate, or urine.

39. The method of claim 37 wherein the target protein is one or more proteins selected from the group consisting of Albumin, IgG, Fibrinogen, Transferrin, IgA, α2-Macroglobulin, IgM, α1-Antitrypsin, Haptoglobin, α1-Acid Glycoprotein, Apolipoprotein A-I and Apolipoprotein A-Il or High Density Lipoprotein.

40. The method of claim 37 wherein the target protein is Fibrinogen.

41. A method of identifying an association between proteins in a biological sample which comprises:
a. providing a biological sample containing a mixture of proteins;
b. contacting the biological sample with a polyclonal IgY antibody composition of claim 3 whereby a desired protein in the complex mixture specifically binds with the polyclonal IgY antibodies in the Fab regions;
c. recovering the desired protein that specifically bound with the polyclonal IgY composition; and
d. analyzing the desired protein to determine if other proteins in the biological sample are associated with the desired protein.

42. The method of claim 41 wherein the desired protein is HSA.

43. A method of preparing a polyclonal IgY antibody composition which comprises contacting reactive polyclonal IgY antibodies, wherein the glycosylation moieties in the Fc region have been oxidized, with a solid support material containing hydrazide moieties wherein the oxidized glycosylation moieties of the polyclonal IgY antibodies covalently bond with hydrazide moieties of the solid support material by forming hydrazone bonds whereby the IgY polyclonal antibodies are oriented to allow the Fab regions to react with an antigen.

44. A polyclonal antibody composition which comprises:
a. polyclonal IgY antibodies having an Fc region and Fab regions
b. a solid support covalently linked with a bond to oxidized glycosylation moieties in the Fc region of the polyclonal IgY antibodies
wherein said polyclonal IgY antibodies are made by immunizing a bird with an antigen present in a cellular system and said antibodies specifically bind with said cellular antigen.

45. A method of affinity separating at least one target in a complex mixture which comprises:
a. providing a complex mixture which contains at least one target,
b. contacting the complex mixture with an affinity separation composition of claim 1 wherein at least one target in the complex mixture specifically binds with the affinity reagent, and
c. recovering the treated complex mixture wherein the concentration of at least one target has been substantially reduced.

46. The method of claim 45 wherein the affinity reagents are IgY polyclonal antibodies having an Fc region and Fab regions, proteins, recombinant proteins, peptides, nucleotides, polymers or a mixture thereof and the target is an antigen.

47. The method of claim 46 wherein the target is a protein, a protein-protein complex, a protein-nucleotide complex, a protein-sugar/lipid complex, a biological complex, a nucleotide a cell, a subcellular organelle, a microorganism or mixtures thereof.

48. The method of claim 45 wherein the target is Albumin, IgG, Fibrinogen, Transferrin, IgA, α2-Macroglobulin, IgM, α1-Antitrypsin, Haptoglobin, α1-Acid Glycoprotein, Apolipoprotein A-I and Apolipoprotein A-II or High-Density Lipoprotein, or mixtures thereof.

49. A method of affinity separating at least one target protein in a complex protein mixture which comprises:
a. providing a complex human protein mixture which contains at least one target human protein,
b. contacting the complex protein mixture with a polyclonal IgY composition of claim 3 wherein at least one target protein in the complex mixture specifically binds with the polyclonal IgY antibodies in the Fab regions and
c. recovering the treated complex protein mixture wherein the concentration of at least one target protein has been substantially reduced.

50. The method of claim 49 wherein the complex protein mixture is plasma, serum, derived from tissue, cell line, cerebrospinal fluid, bronchial alveolar lavage, vitreous humor, nipple aspirate, or urine.

51. The method of claim 49 wherein the target protein is one or more proteins selected from the group consisting of HSA, IgG, Fibrinogen, Tρανσφερριν, IγA, α2-Macroglobulin, IgM, α1-Antitrypsin, Haptoglobin, α1-Acid Glycoprotein, Apolipoprotein A-I and Apolipoprotein A-II or High-Density Lipoprotein.
